(19) United States
(12) Patent Application Publication
(10) Pub. No.: US 2024/0024205 A1
(43) Pub. Date: Jan. 25, 2024

(54) DENTAL RETRACTION COMPOSITION

(71) Applicant: 3M INNOVATIVE PROPERTIES COMPANY, St. Paul, MN (US)

(72) Inventors: Peter U. Osswald, Tuerkheim (DE); Henning Hoffmann, Windach (DE); Paul J. Homnick, Lake Elmo, MN (US); Joachim Zech, Kaufering (DE)

(21) Appl. No.: 18/036,201

(22) PCT Filed: Dec. 1, 2021
(86) PCT No.: PCT/IB2021/061179

§ 371 Date: May 10, 2023

(30) Foreign Application Priority Data
Dec. 11, 2020 (EP) 20213273.4

(51) Int. Cl. A61K 6/18; A61K 6/60; A61P 1/02

(52) U.S. Cl. CPC A61K 6/18; A61K 6/60; A61P 1/02; A61C 9/0033

(57) ABSTRACT

The invention relates to a dental retraction composition comprising guanidinyl-containing polymer, carrageenan, filler, paste-forming component, organic acid having a molecular weight of 80 to 250 g/mol, a pks value of 2 to 5, 1 to 3 carboxylic acid moieties, and being soluble in polyethylene glycol with a molecular weight of 400 g/mol, the dental retraction composition not containing water in an amount of more than 5 wt. %, and not containing aluminium chloride in an amount of more than 0.5 wt. %, wt. % with respect to the weight of the dental retraction composition. The dental retraction composition is for use in a process for use in a process of retracting dental tissue in combination with coagulating blood. The invention also relates to a delivery device comprising the dental retraction composition and a kit of parts comprising the delivery device, an applier for the delivery device, optionally a dental impression material, and optionally a retraction cap.

### FIELD OF THE INVENTION

The invention relates to a dental retraction composition comprising a guanidinyl-containing polymer, carrageenan and at least one organic acid. The retraction paste has blood coagulating and moisture-absorbing properties. The paste expands upon contact with water.

### BACKGROUND

For producing of well-fitting dental crowns and bridges, it is typically necessary to first record the current dental situation in the mouth of a patient.

This can be done by using dental impression materials or by scanning the surface of the dental situation in the mouth of the patient, in particular the surface of the prepared tooth or teeth to be reconstructed.

However, besides the necessity to record the immediate visible surface of the dental situation it is also typically necessary to record the so-called preparation margin of the prepared tooth. The preparation margin is typically close to the gumline and may not be easily visible or recordable.

For better access to the preparation margin often a so-called retraction procedure is suggested where a part of the gingiva is temporarily removed from the hard dental structure.

For retracting gingiva from a prepared tooth a cord can be used. In this respect, a retraction cord is packed between gingival tissue and the margin of the prepared tooth (this region is also often called sulcus) using an appropriate dental instrument. To obtain sufficient vertical and horizontal retraction of gingival tissue, it is often necessary to pack several lengths of retraction cord into the sulcus in order to be able to make a detailed dental impression.

A description of the background in regard to retraction cords can be found e.g. in U.S. Pat. No. 4,522,593 (Fischer). Generally, dental retraction cords are sometimes difficult to place into the gingival sulcus. The procedure can also be time consuming. It can also be cumbersome to remove the retraction cord prior to taking the impression. Coagulated blood may adhere to the cord and removing it may open the wound again which results in bleeding.

For a more convenient placement retraction pastes have been suggested.

A commercially available product to be used for retraction is sold under the name Expasyl™. However, it has been reported, that Expasyl™ is only effective under specific, limited conditions when the sulcus is flexible and has a sufficient depth. The paste's thickness makes it difficult for some practitioners to express it into the sulcus. Moreover, according to the instruction of use, the viscosity of the composition might change when fluids like water, saliva or blood are absorbed.

Generally, removing non-hardening pastes completely out of the sulcus before taking a dental impression can be very time consuming and cumbersome. Usually, the paste is rinsed off using water-spray. However, sometimes paste residues are located deep in the sulcus and are thus difficult to remove. These residues might prevent the impression material from flowing into the sulcus area and may negatively influence the setting of the impression material which is subsequently applied. Moreover, after rinsing off the paste with water an additional drying step might be required before the impression can be taken. These removing and drying steps could cause bleeding of the tissue and might make an impression taking step more complicated.

Hardening materials are sometimes easier to remove. However, they are not very hydrophilic. This might cause problems with regard to flowability of the material into the gingival sulcus.

US 2008/0220050 A1 (Chen) relates to a composition for gingival retraction. The pasty composition contains water, clay, glass filler and astringent.

WO 2006/057535 A1 (Kim) describes a composition comprising a certain amounts of kaolin clay, water, aluminium chloride hexahydrate, starch powder, silicone oil and colouring agent.

US 2005/008583 A1 (White) describes a gingival retraction material comprising a carrying medium, a retraction medium and an anti-evaporating component. As an example the following material formula is given: kaolin powder (80 wt. %), aluminium chloride (15 wt. %), water/glycerine sufficient to produce a heavy plastic consistency, flavourings/colour as desired.

US 2012/077142 A1 (Maurer et al.) describes a retraction material containing a mixture of layer type 1:1 silicate filler and a layer type 2:1—silicate mineral filler in a ratio 50/50 to 5/95 wt. % as filler. Additional optional ingredients are water, aluminium chloride as astringent as well as silicone oil. In addition the application describes a capsule as application system for delivering the composition into the sulcus.

US 2014/0348921 A1 (Lesage) describes a retraction paste containing an astringent, preferably aluminium chloride, clay, a texturing agent like carrageenan, water (50-70%), and humectant like polyethylene glycol. It is stated that these pastes allow a slower release of aluminium, are less water-sensible and thus allow longer treatment times. But as consequence the rinsability of these pastes is significantly reduced.

US 2014/0348921 A1 (Lesage) describes a haemostatic composition in the form of an extrudable hydrophilic paste, characterized in that it contains, expressed as percentage by weight relative to the total weight of said composition: between 5 and 15% of an astringent, preferably aluminium chloride; between 2 and 15% of kaolinitic clay, preferably kaolin; between 10 and 20% of a texturizing agent comprising one (or more) naturally-occurring polysaccharide(s) with gelling or thickening power optionally combined with plant fibres; between 50 and 70% of water; between 0 and 20% of a humectant.

EP 3 574 887 A1 (Voco) describes a retraction paste containing chitosan in combination with tartaric acid to enable aluminium free pastes.

US 2009/0042170 A1 (Chen et al.) describes a method for temporarily widening a gingival sulcus, the method comprising: inserting a composition within a gingival sulcus to be widened, and thereafter photo curing to polymerize the composition, the composition comprising a polymerizable monomer having at least one ethylenically unsaturated group, a photo polymerization initiator, and a fine inorganic powder, the method temporarily widening the gingival sulcus.

US 2018/0064844 A1 (Dahl et al.) describes a hemostatic paste comprising a hemostatic component and a light-curable component as well as a method for providing hemostasis to a wound.

US 2019/0282453 A1 (Hoffmann et al.) describes a medical composition comprising guanidinyl-containing polymer(s) and polyanionic polymer(s). The medical composition is useful for absorbing water containing fluids and can be used as dental retraction composition or as part of a medical treatment device.

However, there is still room for improvement especially with regard to the requirements to be fulfilled with respect to modern dental materials.

### DESCRIPTION OF THE INVENTION

There is still a need for an improved dental composition, which can be used as dental retraction material.

The dental retraction material should have sufficient capability to absorb fluids, in particular water containing or water-based fluids, like blood.

The dental composition should have blood coagulating or astringent properties, as well.

The dental composition should also be sufficiently storage stable.

It would also be desirable, if the dental composition can be applied easily to the sulcus of a tooth, e.g. expressed through a small or thin nozzle.

The dental composition should also be easily removable from the sulcus, either by rinsing with water or by using e.g. a tweezer or probe.

One or more of the above objects are addressed by the invention described in the present text.

In one embodiment the present invention features a dental retraction composition comprising guanidinyl-containing polymer, carrageenan, filler, paste-forming component, organic acid having a molecular weight of 80 to 250 g/mol, a pks value of 2 to 5, 1 to 3 carboxylic acid moieties, and being soluble in polyethylene glycol with a molecular weight of 400 g/mol, the dental retraction composition not containing water in an amount of more than 5 wt. %, and not containing aluminium chloride in an amount of more than 0.5 wt. %, wt. % with respect to the weight of the dental retraction composition.

The dental retraction composition as described in the present text is for use in a process for use in a process of retracting dental tissue in combination with coagulating blood, the process comprising the steps of applying the dental retraction composition into the sulcus of a prepared tooth, leaving the dental retraction composition in the sulcus for a time sufficient to absorb moisture and to coagulate blood being present in the sulcus, optionally applying light to the dental retraction composition, removing the dental retraction composition from the sulcus.

The invention also relates to a delivery device comprising the dental retraction composition as described in any of the present text, the delivery system having the shape of a capsule, compule, syringe or cartridge.

The invention further relates to a kit of parts comprising the delivery device comprising the dental retraction composition as described in the present text, an applier for the delivery device, optionally a dental impression material, and optionally a retraction cap.

Unless otherwise specified, within the context of the text of the invention, the following terms have the following meanings.

A “water absorbing composition” is a composition being able to absorb water in an amount of at least 50 wt. % or at least 100 wt. % or at least 200 wt. % with respect to the weight of the composition.

A “dental composition” is a composition which can or is to be used in the dental or orthodontic field.

A “dental retraction composition” is a composition enabling the practitioner to retract soft dental tissue (e.g. gingiva) away from hard dental tissue (e.g. tooth) before or during an impression of the tooth structure is made.

A “tooth structure” is any tooth structure, prepared or ready for preparation by the dentist. It can be a single tooth or two or more teeth. A tooth structure is also referred to as hard dental tissue in contrast to soft dental tissue (e.g. gingiva).

A “dental impression material” is a material used for making impressions of the tooth structure including the gingiva. A dental impression material is usually applied on a dental impression tray. A dental impression material can be based on different chemical substances and crosslink by various chemical reactions (including addition curing and condensation curing materials). Typical examples include silicone based impression materials (e.g. VPS materials) and polyether based impression materials and mixtures of those.

A “liquid” is any solvent or liquid which is able to at least partially disperse, dissolve or suspend the components being present in the composition at ambient conditions (e.g. 23° C.).

A “paste” is a material that typically consist of a suspension of granular material in a liquid. Pastes can be classified by their viscosity or their consistency comparable to dental impression material (cf. ISO 4823).

A “haemostatic agent” is an agent which is able to reduce bleeding to a certain amount and/or causes blood to coagulate. Haemostatic agents are also sometimes referred to as astringents.

A “particle” means a substance being a solid having a shape which can be geometrically determined. The shape can be regular or irregular. Particles can typically be analysed with respect to e.g. grain size and grain size distribution.

If desired, the particle size can be measured using a Cilas 1064 LD Nass (Cilas, France) light scattering instrument. The Cilas 1064 uses an integrated optical system to cover the range from 0.04 to 500 m. The mixtures to be analyzed are added to the test chamber filled with water. Ultrasound is applied for about 60 s in order not to alter the particle size distributions and to avoid agglomeration. The raw data is processed with the instrument software using the Fraunhofer approximation, frequently used techniques known to the expert in the art.

“Phyllosilicates” are silicates forming sheets of silicate tetrahedra with Si₂O₅. Phyllosilicates can be further divided in sub-groups, e.g. according to the number of sheets or layers arranged with each other.

Within the meaning of the present text, phyllosilicates are divided in the following subgroups: silicate minerals of the 2:1 layer type group and silicate minerals of the 1:1 layer type group.

Clay minerals belong to the group of phyllosilicates can be characterized by the number of layers linked or arranged with each other. This classification is also used in the present text.

E.g., in kaolinite, having the ideal formula Al₂[Si₂O₅(OH)₄]), two single layers are linked or arranged with each other.

E.g. in muscovite, having the ideal formula KAl₂(AlSi₃O₁₀)(OH)₂ and belonging to the mica type group of minerals, three layers are linked or arranged with each other.

The terms “crosslinking”, “hardening”, “setting”, “curing” or “curable” are used interchangeable, all referring to the formation of material with a higher molecular weight and/or to the formation of a material having a higher viscosity, by creating a network due to chemical and/or physical interaction.

A “hardening-”, “curing-” or “setting-reaction” is a reaction, wherein physical properties such as viscosity, and tensile strength of a composition change over the time due to a chemical or physical reaction between the individual components.

“Radiation curable” means that the component (or composition, as the case may be) can be cured by applying radiation, preferably electromagnetic radiation with a wavelength in the visible light spectrum (380 to 740 nm) under ambient conditions and within a reasonable time frame (e.g. within about 60, 30 or 10 s).

A “hydrophilic component” is soluble not only in water (typically by more than 1 or 5 or 10 wt. %) but also in other polar solvents.

A composition or solution is “essentially or substantially free of” a certain component, if the composition does not contain said component as an essential feature. Thus, said component is not wilfully added to the composition either as such or in combination with other components or ingredient of other components. A composition being essentially free of a certain component usually contains the component in an amount of less than about 1 wt. % or less than about 0.1 wt. % or less than about 0.01 wt. % with respect to the whole composition. Ideally the composition does not contain the said component at all. However, sometimes the presence of a small amount of the said component is not avoidable e.g. due to impurities.

“Ambient conditions” mean the conditions which the dental retraction composition is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically 20 to 25° C. and 1,000 to 1,025 mbar (adjusted to sea level).

If not indicated otherwise “molecular weight” always means Mw (weight average of the molecular weight) and can be determined for the individual classes of polymers by gel permeation chromatography (GPC) against a standard of defined molecular weight. Suitable measurement methods are known to the person skilled in the art.

If not indicated otherwise, wt. % always refers to the weight of the whole composition. As used herein, “a”, “an”, “the”, “at least one” and “one or more” are used interchangeably.

The terms “comprise” or “contain” and variations thereof do not have a limiting meaning where these terms appear in the description and claims. The term “comprise” shall include also the terms “consist essentially of” and “consists of”.

Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

### DETAILED DESCRIPTION

It was found that the dental retraction composition comprising a combination of guanidinyl-containing polymer(s), carrageenan(s) and organic acids shows an enhanced capacity of absorbing fluid, like water or water-based fluids and has also adequate blood coagulating properties.

When the dental retraction composition comes in contact with water, the composition swells to a multiple of its original volume and forms a soft structure. This structure can be easy removed, even if located in a sulcus of a tooth.

Without wishing to be bound to a certain theory, it seems that there is a synergistic effect if a guanidinyl-containing polymer is used in combination with carrageenan(s).

Further, the use of guanidinyl-containing polymer(s) in the dental retraction composition enables the formulation of a composition with improved storage stability, in particular if the composition is for use as dental retraction composition.

Currently, most dental retraction compositions contain water for dissolving or dispersing other components contained in the composition.

During storage water may evaporate from the composition which typically causes a non-desired increase of the viscosity of the composition.

High viscous compositions typically require a higher force for distributing the composition from a packaging device. In fact, the distribution of a high viscous composition through a thin nozzle into the sulcus of a tooth may become nearly impossible.

To avoid a non-wanted evaporation of water from the composition, the composition has either to be stored under specific conditions (e.g. contained in a sealed blister) or a reduced shelf-life has to be accepted.

It was found that due to the use of guanidinyl-containing polymer(s), the dental retraction composition described in the present text can be formulated essentially without using or adding water. Thus, the dental retraction composition described in the present text does not contain water in an amount of more than 5 wt. %.

The risk of a non-desired increase of the viscosity due to evaporation of a solvent is thus reduced. Thus, a more storage stable composition can be provided.

Further, the dental retraction composition can be stored in conventional packaging materials without the need for an additional sealed pouch.

In addition, it was found that the flow resistance of the dental retraction composition can be improved, if guanidinyl-containing polymer(s) were used or added.

Moreover, it was found that, if the dental retraction composition is stored in a specific container with a cannula or nozzle having certain dimensions, the composition can be applied to a sulcus using a commercially available dispensing device with acceptable extrusion forces (e.g. less than or equal to about 150 N).

Finally, due to the astringent properties of the guanidinyl-containing polymer(s), the amount of further, more aggressive astringents like aluminium salts (e.g. aluminium chloride) or other heavy metal salts which are typically used for this purpose can be reduced or avoided at all.

Being able to provide a dental retraction composition containing a reduced amount or being essentially free of aluminium chloride or other heavy metal salts as astringents, can be beneficial as this may contribute to a better compatibility of the dental retraction composition with packaging materials.

Further, it was found that the addition of organic acids having certain properties supports the astringent properties of the composition without negatively affecting other properties like storage stability or rheological behavior.

It was also found that, if desired, the composition can be formulated as a curable composition by adding radically curable components without negatively affecting its absorption properties.

Adding radically curable components in combination with an initiator system may facilitate the removal of the cured composition in one piece after curing.

Thus, a more intense rising step which may cause additional bleeding can be avoided.

The dental retraction composition comprises a guanidinyl-containing polymer.

It was found that using a guanidinyl-containing polymer allows the formulation of a dental retraction composition with either a reduced amount of or even without an inorganic astringent such as aluminium chloride.

Alternatively or in addition, the formulation of compositions being essentially free of added water is now possible.

Further, using a guanidinyl-containing polymer may help to reduce the extrusion force and/or flow resistance of the dental retraction composition.

The term “guanidinyl-containing polymer” includes also polymers where the guanidinyl moiety is present in its protonated form including the salts thereof (in particular chloride and sulphate salts).

Suitable polymers include polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), poly(acrylamide-co-aminoethylmethacrylate), polyethylenimine, polypropylenimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or copolymers, amino-containing polyacrylate homo- or copolymers.

For some embodiments, the preferred amino-containing polymers include polyaminoamides, polyethyleneimine, polyvinylamine, polyallylamine, polydiallylamine and acrylamide-based polymers.

As used herein, the term “guanidinyl” refers to a group of the following formula

—NR³—C(═NR⁴)—NR⁴R⁵.

If the guanidinyl group is part of a pendant group, the group R³ refers to hydrogen, C₁-C₁₂ (hetero)alkyl, or C₅-C₁₂ (hetero)aryl.

If the guanidinyl group is part of the backbone of the polymer, the group R³ can refer to a residue of a polymer chain.

Each group R⁴ is independently hydrogen, C₁-C₁₂ (hetero)alkyl, or C₅-C₁₂ (hetero)aryl. Group R⁵ is hydrogen, C₁-C₁₂ (hetero)alkyl, C₅-C₁₂ (hetero)aryl, or a group of formula —N(R⁴)₂.

The guanidinyl group can be part of a biguanidinyl group that is of formula —NR³—C(═NR⁴)—NR⁴—C(═NR⁴)—NR⁴R⁵ where the groups R³, R⁴, and R⁵ are the same as defined above.

Although any guanidinyl-containing polymer can be used in the cationic form, this polymer is often of Formula (I).

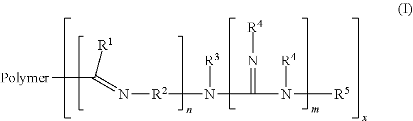

In Formula (I), the group R¹ is hydrogen, C₁-C₁₂ (hetero)alkyl, or C₅-C₁₂ (hetero)aryl, or a residue of the polymer chain. The group R² is a covalent bond, a C₂-C₁₂ (hetero)alkylene, or a C₅-C₁₂ (hetero)arylene. The group R³ is H, C₁-C₁₂ (hetero)alkyl, or C₅-C₁₂ (hetero)aryl, or can be a residue of the polymer chain when n is 0. Each group R⁴ is independently hydrogen, C₁-C₁₂ (hetero)alkyl, or C₅-C₁₂ (hetero)aryl. The group R⁵ is hydrogen, C₁-C₁₂ (hetero)alkyl, C₅-C₁₂ (hetero)aryl, or —N(R⁴)₂. The variable n is equal to 0 or 1 depending on the precursor polymer used to form the guanidinyl-containing polymer. The variable m is equal to 1 or 2 depending on whether the cationic group is a guanidinyl or biguanidinyl group. The term “Polymer” in Formula (I) refers to all portions of the guanidinyl-containing polymer except the x groups of formula —[C(R′)═N—R²—]ₙN(R³)—[C(═NR⁴)—NR⁴R⁵—]ₘ. The term x is a variable equal to at least 1 or in a range of 10 to 1,000 or 100 to 1,000.

Most guanidinyl-containing polymers have more than one guanidinyl group. The number of guanidinyl groups can be varied depending the method used to prepare the guanidinyl-containing polymer. For example, the number of guanidinyl groups can depend on the choice of precursor polymer selected for reacting with a suitable guanylating agent. In some embodiments, the variable x can be up to 1000, up to 500, up to 100, up to 80, up to 60, up to 40, up to 20, or up to 10.

The guanidinyl-containing polymer of Formula (I) is often the reaction product of (a) a precursor polymer and (b) a suitable guanylating agent.

The precursor polymer is often an amino-containing polymer or a carbonyl-containing polymer. When the precursor polymer is an amino-containing polymer, the variable n in Formula (I) is typically equal to 0. When the precursor polymer is a carbonyl-containing polymer, the variable n is equal to 1. If the guanylating agent contains a guanidinyl group or a precursor of a guanidinyl group, the variable m in Formula (I) is equal to 1. If the guanylating agent contains a biguanidinyl group or a precursor of a biguanidinyl group, the variable m in Formula (I) is equal to 2.

In embodiments where n is 0, the base polymer of the guanidinyl-containing polymer is often prepared by reaction of a suitable guanylating agent and an amino-containing polymer. In other embodiments, where n is 1, the guanidinyl-containing polymer is often prepared by reaction of a suitable guanylating agent and a carbonyl-containing polymer.

In those embodiments where n is 0 and the precursor polymer is an amino-containing polymer, the structure of the guanidinyl-containing polymer of Formula (I) can also be written more simply as the structure of Formula (II).

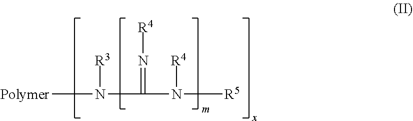

In Formula (II), the group R³ is hydrogen, C₁-C₁₂ (hetero)alkyl, or C₅-C₂ (hetero)aryl, or can be a residue of the polymer chain. When the guanidinyl group is part of a pendant group, R³ is hydrogen, C₁-C₁₂ (hetero)alkyl, or C₅-C₁₂ (hetero)aryl. Each R⁴ is independently hydrogen, C₁-C₁₂ (hetero)alkyl, or C₅-C₁₂ (hetero)aryl. The group R⁵ is hydrogen, C₁-C₁₂ (hetero)alkyl, or C₅-C₁₂ (hetero)aryl, or —N(R⁴)₂. The variable in is equal to 1 or 2. The term “Polymer” in the formula above refers to all portions of the guanidinyl-containing polymer except the x groups of formula —N(R³)—[C(═NR⁴)—NR⁴R⁵—]ₘ. The term x is a variable equal to at least 1 or in a range of 10 to 1,000 or 100 to 1,000.

The amino-containing polymer used as a precursor polymer to prepare a guanidinyl-containing polymer of Formula (II) can be represented by the formula Polymer N(R³)H. As noted above, however, the amino-containing polymer typically has many groups —N(R³)H but Formula (I) shows only one for ease of discussion purposes only. The —N(R³)H groups can be a primary or secondary amino group and can be part of a pendant group or part of the backbone of the precursor polymer. The amino-containing polymers can be synthesized or can be naturally occurring biopolymers. Suitable amino-containing polymers can be prepared by chain growth or step growth polymerization procedures with amino-containing monomers. These monomers can also, if desired, be copolymerized with other monomers without an amino-containing group. Additionally, the amino-containing polymers can be obtained by grafting primary or secondary amine groups using an appropriate grafting technique.

The guanidinyl-containing polymer also includes polymers where the guanidinyl moiety is protonated including polymers having the following formula:

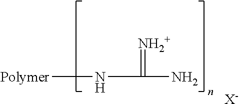

with X⁻ being selected from Cl⁻, Br⁻, I⁻, ½SO₄²⁻, NO₃⁻, CH₃COO⁻, C₃H₇COO⁻ and n being a variable equal to at least 1 or in a range of 10 to 1,000 or 100 to 1,000.

Examples of amino-containing polymers suitable for use, which are prepared by chain growth polymerization include, but are not limited to, polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), and poly(acrylamide-co-aminoethylmethacrylate).

Examples of amino-containing polymers suitable for use, which are prepared by step growth polymerization include, but are not limited to, polyethylenimine, polypropylenimine, polylysine, polyaminoamides, polydimethylamine-epichlorohydrin-ethylenediamine, and any of a number of polyaminosiloxanes, which can be prepared from monomers such as aminopropyltriethoxysilane, N-(2-aminoethyl)-3-aminopropyltrimethoxysilane, N-trimethoxysilylpropyl-N-methylamine, and bis(tri-methoxysilylpropyl)amine.

Other useful amino-containing polymers that have primary or secondary amino end groups include, but are not limited to, dendrimers (hyperbranched polymers) formed from polyamidoamine (PAMAM) and polypropylenimine. Exemplary dendrimeric materials formed from PAMAM are commercially available under the trade designation “STARBURST (PAMAM) dendrimer” (e.g., Generation 0 with 4 primary amino groups, Generation 1 with 8 primary amino groups, Generation 2 with 16 primary amino groups, Generation 3 with 32 primary amino groups, and Generation 4 with 64 primary amino groups) from Aldrich Chemical (Milwaukee, WI). Dendrimeric materials formed from polypropylenimine are commercially available under the trade designation “DAB-Am” from Aldrich Chemical. For example, DAB-Am-4 is a generation 1 polypropylenimine tetraamine dendrimer with 4 primary amino groups, DAB-Am-8 is a generation 2 polypropylenimine octaamine dendrimer with 8 primary amino groups, DAB-Am-16 is a generation 3 polypropylenimine hexadecaamine with 16 primary amino groups, DAB-Am-32 is a generation 4 polypropylenimine dotriacontaamine dendrimer with 32 primary amino groups, and DAB-Am-64 is a generation 5 polypropylenimine tetrahexacontaamine dendrimer with 64 primary amino groups.

Examples of suitable amino-containing polymers that are biopolymers include chitosan as well as starch that is grafted with reagents such as methylaminoethylchloride.

Still other examples of amino-containing polymers include polyacrylamide homo- or copolymers and amino-containing polyacrylate homo- or copolymers prepared with a monomer composition containing an amino-containing monomer such as an aminoalkyl(meth)acrylate, (meth)acrylamido-alkylamine, and diallylamine.

For some embodiments, the preferred amino-containing polymers include polyaminoamides, polyethyleneimine, polyvinylamine, polyallylamine, and polydiallylamine.

Suitable commercially available amino-containing polymers include, but are not limited to, polyamidoamines that are available under the trade designations ANQUAMINE (e.g., ANQUAMINE 360, 401, 419, 456, and 701) from Air Products and Chemicals (Allentown, PA), polyethylenimine polymers that are available under the trade designation LUPASOL (e.g., LUPASOL FG, PR 8515, Waterfree, P, and PS) from BASF Corporation (Resselaer, NY), polyethylenimine polymers such as those available under the trade designation CORCAT P-600 from EIT Company (Lake Wylie, SC), and polyamide resins such as those available from Cognis Corporation (Cincinnati, OH) under the traded designation VERSAMID series of resins that are formed by reacting a dimerized unsaturated fatty acid with alkylene polyamines.

Guanidinyl-containing polymers can be prepared by reaction of the amino-containing polymer precursor with a guanylating agent.

Although all the amino groups of the amino-containing polymer can be reacted with the guanylating agent, there are often some unreacted amino groups from the amino-containing polymer precursor remaining in the guanidinyl-containing polymer. Typically, at least 0.1 mole percent, at least 0.5 mole percent, at least 1 mole percent, at least 2 mole percent, at least 10 mole percent, at least 20 mole percent, or at least 50 mole percent of the amino groups in the amino-containing polymer precursor are reacted with the guanylating agent. Up to 100 mole percent, up to 90 mole percent, up to 80 mole percent, or up to 60 mole percent of the amino groups can be reacted with the guanylating agent. For example, the guanylating agent can be used in amounts sufficient to functionalize 0.1 to 100 mole percent, 0.5 to 90 mole percent, 1 to 90 mole percent, 1 to 80 mole percent, 1 to 60 mole percent, 2 to 50 mole percent, 2 to 25 mole percent, or 2 to 10 mole percent of the amino groups in the amino-containing polymer.

Known guanylating agents for reaction with an amino-containing polymer precursor include, but are not limited to, cyanamide; O-alkylisourea salts such as O-methylisourea sulfate, O-methylisourea hydrogen sulfate, O-methylisourea acetate, O-ethylisourea hydrogen sulfate, and O-ethylisourea hydrochloride; chloroformamidine hydrochloride; 1-amidino-1,2,4-triazole hydrochloride; 3,5-dimethylpyrazole-1-carboxamidine nitrate; pyrazole-1-carboxamidine hydrochloride; N-amidinopyrazole-1-carboxamidine hydrochloride; and carbodiimides such as dicyclohexylcarbodiimide, N-ethyl-N′-(3-dimethylaminopropyl)carbodiimide, and diisopropylcarbodiimide. The amino-containing polymer may also be acylated with guanidino-functional carboxylic acids such as guanidinoacetic acid and 4-guanidinobutyric acid in the presence of activating agents such as EDC (N-[3-(dimethylamino)propyl]-3-ethylcarbodiimide hydrochloride), or EEDQ (2-ethoxy-1-ethoxycarbonyl-1,2-dihydroquinoline). Additionally, the guanidinyl-containing polymer may be prepared by alkylation with chloroacetone guanyl hydrazone, e.g. as described in U.S. Pat. No. 5,712,027 (Ali et al.).

Guanylating agents for the preparation of biguanide-containing polymers include sodium dicyanamide, dicyanodiamide and substituted cyanoguanidines such as N³-p-chlorophenyl-N¹-cyanoguanidine, N³-phenyl-N¹-cyanoguanidine, N³-alpha-naphthyl-N′-cyanoguanidine, N³-methyl-N¹-cyanoguanidine, N³,N³-dimethyl-N¹-cyanoguanidine, N³-(2-hydroxyethyl)-N′-cyanoguanidine, and N³-butyl-N¹-cyanoguanidine. Alkylene- and arylenebiscyanoguanidines may be utilized to prepare biguanide functional polymers by chain extension reactions. The preparation of cyanoguanidines and biscyanoguanidines is described in detail in Rose, F. L. and Swain, G. J. Chem Soc., 1956, pp. 4422-4425. Other useful guanylating reagents are described e.g. by Alan R. Katritzky et al., Comprehensive Organic Functional Group Transformation, Vol. 6, p. 640.

The guanidinyl-containing polymer formed by reaction of an amino-containing polymer precursor and a guanylating agent will have pendent or catenary guanidinyl groups of the Formula (III).

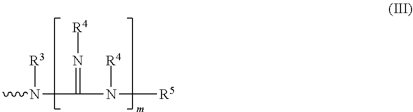

In Formula (III), the groups R³, R⁴, and R⁵ and the variable m are the same as defined above. The wavy line attached to the N(R³) group shows the position of attachment the group to the rest of the polymeric material. In most embodiments, the group of Formula (III) is in a pendant group of the guanidinyl-containing polymer.

In some embodiments, it may be advantageous to react the amino-containing polymer precursor to provide other ligands or groups in addition to the guanidinyl-containing group. For example, it may be useful to include a hydrophobic ligand, an ionic ligand, or a hydrogen bonding ligand. This can be particularly advantageous for the removal of certain microorganisms during the wiping of a microorganism-contaminated surface.

The additional ligands can be readily incorporated into the amino-containing polymers by alkylation or acylation procedures well known in the art. For example amino groups of the amino-containing polymer can be reacted using halide, sulfonate, and sulfate displacement reactions or using epoxide ring opening reactions. Useful alkylating agents for these reactions include, for example, dimethylsulfate, butyl bromide, butyl chloride, benzyl bromide, dodecyl bromide, 2-chloroethanol, bromoacetic acid, 2-chloroethyltrimethylammonium chloride, styrene oxide, glycidyl hexadecyl ether, glycidyltrimethylammonium chloride, and glycidyl phenyl ether. Useful acylating agents include, for example, acid chlorides and anhydrides such as benzoyl chloride, acetic anhydride, succinic anhydride, and decanoyl chloride, and isocyanates such as trimethylsilylisocyanate, phenyl isocyanate, butyl isocyanate, and butyl isothiocyanate. In such embodiments 0.1 to 20 mole percent, preferably 2 to 10 mole percent, of the available amino groups of the amino-containing polymer may be alkylated and/or acylated.

The guanidinyl-containing polymer can be crosslinked. The amino-containing polymer can be crosslinked prior to reaction with the guanylating agent. Alternatively, the guanidinyl-containing polymer can be crosslinked by reaction of a crosslinker with remaining amino groups from the amino-containing polymer precursor or with some of the guanidinyl groups. Suitable crosslinkers include amine-reactive compounds such as bis- and polyaldehydes such as glutaraldehyde, bis- and polygylcidylethers such as butanedioldiglycidylether and ethyleneglycoldiglycidylether, polycarboxylic acids and their derivatives (e.g., acid chlorides), polyisocyanates, formaldehyde-based crosslinkers such as hydroxymethyl and alkoxymethyl functional crosslinkers, such as those derived from urea or melamine, and amine-reactive silanes, such as 3-glycidoxypropyltrimethoxysilane, 3-glycidoxypropyltriethoxysilane, 5,6-epoxyhexyltriethoxysilane, (p-chloromethyl)phenyltrimethoxysilane, chloromethyltriethoxysilane, 3-isocyanatopropyltriethoxysilane, and 3-thiocyanatopropyltriethoxysilane.

In other embodiments, the guanidinyl-containing polymer is of Formula (IV), which corresponds to Formula (I) where n is equal to 1.

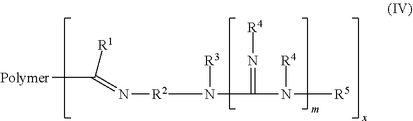

In Formula (IV), the group R¹ is hydrogen, C₁-C₁₂ (hetero)alkyl, or C₅-C₁₂ (hetero)aryl, or a residue of the polymer chain. If the guanidinyl-containing group is the reaction product of a guanylating agent and a carbonyl group that is part of the backbone of the polymer, R¹ is a residue of the polymer chain. Group R² is a covalent bond, a C₂-C₁₂ (hetero)alkylene, or a C₅-C₁₂ (hetero)arylene. Group R³ is hydrogen, C₁-C₁₂ (hetero)alkyl, or C₅-C₁₂ (hetero)aryl. Each R⁴ is independently H, C₁-C₁₂ (hetero)alkyl, or C₅-C₁₂ (hetero)aryl. Group R⁵ is hydrogen, C₁-C₁₂ (hetero)alkyl, or C₅-C₁₂ (hetero)aryl, or —N(R⁴)₂. The variable m is equal to 1 or 2. The term “Polymer” in Formula (I) refers to all portions of the guanidinyl-containing polymer except the x groups of formula —C(R′)═N—R²—N(R³)—[C(═NR⁴)—NR⁴R⁵-]. The term x is a variable equal to at least 1 or in a range of 10 to 1,000 or 100 to 1,000.

Guanidinyl-containing polymers of Formula (IV) are the reaction product of a carbonyl-containing polymer and a suitable guanylating agent for reaction with a carbonyl group. The carbonyl-containing polymer used as a precursor polymer to prepare a guanidinyl-containing polymer of Formula (IV) can be represented by the formula Polymer-C(O)—R¹. The carbonyl-containing polymer precursor typically has many groups —C(O)—R¹ but Formula (IV) shows only one for ease of discussion purposes only. The carbonyl group —C(O)—R¹ is an aldehyde group (when R¹ is hydrogen) or a ketone group (when R¹ is a (hetero)alkyl or (hetero)aryl). Although the carbonyl-group can be part of the polymeric backbone or part of a pendant group from the polymeric backbone, it is typically in a pendant group.

If desired, the guanidinyl-containing polymers can be produced as described in US 2016/0115430 A1 (Swanson et al.), in particular in sections [0049] to [0080], the description of which is herewith incorporated by reference. If desired, the reaction can be monitored by ¹H-NMR spectroscopy, in particular the chemical shift difference between the primary amine and the guanylated primary amine.

The guanidinyl-containing polymer is typically present in the following amounts: Lower limit: at least 1 or at least 2 or at least 5 wt. %; Upper limit: utmost 60 or utmost 40 or utmost 30 wt. %; Range: from 1 to 60 or from 2 to 40 or from 5 to 30 wt. %; wt. % with respect to the dental retraction composition.

Carrageenans or carrageenins are a family of sulphated polysaccharides that are typically extracted from red edible seaweeds.

There are three main varieties of carrageenan, which differ in their degree of sulfphation.

Kappa-carrageenan has one sulphate group per disaccharide. Iota-carrageenan has two sulphates per disaccharide. Lambda carrageenan has three sulphates per disaccharide. Other carrageenan(s) which are known are epsilon and μ.

With respect to the present text, the use of iota or lambda carrageenan(s) can sometimes be preferred.

Carrageenans are large, highly flexible molecules that curl forming helical structures. This gives them the ability to form a variety of different gels at room temperature.

Carrageenans are polysaccharides made up of repeating galactose units and 3,6 anhydrogalactose (3,6-AG), both sulfated and non-sulfated. The units are typically joined by alternating α-1,3 and β-1,4 glycosidic linkages.

If desired, the carrageenan(s) can be characterized by one or more of the following features:
molecular weight (Mw; weight average): 10,000 to 1,000,000 or 20,000 to 500,000 g/mol;
ester sulphate content: 25 to 40 wt. % or 25 to 30 wt. % with respect to the weight of the carrageenan.

The carrageenan(s) is typically present in the following amounts: Lower limit: at least 1 or at least 2 or at least 5 wt. %; Upper limit: utmost 40 or utmost 35 or utmost 30 wt. %; Range: from 1 to 40 or from 2 to 35 or from 5 to 30 wt. %; wt. % with respect to the dental retraction composition. The ratio of guanidinyl-containing polymer(s) to carrageenan(s) is typically from 4 to 1 to 1 to 4 with respect to weight.

According to one embodiment, the guanidinyl-containing polymer and the carrageenan component are used in essentially equal amounts with respect to weight.

A suitable dental retraction composition may comprise the guanidinyl-containing polymer in an amount of 10 to 30 wt. % and the carrageenan component in an amount of 10 to 30 wt. %.

The dental retraction composition also comprises filler(s).

A wide variety of inorganic, hydrophilic or hydrophobic fillers may be employed such as silicates, silicas (including quartz and cristobalite), alumina, magnesia, titania, inorganic salts, metallic oxides and glasses.

The sizes and surface areas of the filler particles can be adjusted to control the viscosity and thixotropicity of the resulting compositions. Some or all of the fillers may be superficially treated with one or more silanating agents, as known to those of ordinary skill in the art. Such silanating may be accomplished through use of known halogenated silanes or alkoxysilanes or silazanes.

A combination of reinforcing and non-reinforcing fillers can also be used.

In this respect, the amount of reinforcing fillers can range from 1 to 10 wt. %, in particular from 2 to 5 wt. % with respect to the whole composition.

Typical reinforcing fillers include fumed silica, and the like. Pyrogenically-prepared highly-disperse silicic acids which have preferably been hydrophobized by surface treatment are sometimes preferred as reinforcing fillers. The surface treatment can be carried out, for example with dimethyldichlorosilane, hexamethyldisilazane, tetramethylcyclotetrasiloxane or polymethylsiloxane.

Typical non-reinforcing fillers are phyllosilicates, quartz, cristobalite, precipitated silicas, diatomaceous earth, alumina, magnesia, titanium dioxide, zirconium silicate, metallic oxides, barium sulphate, calcium carbonate, plaster, glass and the like.

The non-reinforcing fillers can be surface treated, e.g. silanated, or non-surface treated.

Typical average particle sizes are from 2 to 10 μm.

If present, the filler(s) are typically present in the following amounts: Lower limit: at least 3 or at least 5 or at least 10 wt. %; Upper limit: utmost 70 or utmost 60 or utmost 50 wt. %; Range: from 3 to 70 or from 5 to 60 or from 10 to 50 wt. %; wt. % with respect to the dental retraction composition.

The use of phyllosilicates as filler(s) is sometimes preferred. The nature and structure of the phyllosilicate(s) is not particularly limited unless the desired result cannot be achieved.

Phyllosilicates which can be used include layer type 1:1 silicate minerals, layer type 1:2 silicate minerals and mixtures of layer type 1:1 silicate minerals and layer type 1:2 silicate minerals.

Phyllosilicates from the layer type 1:1 silicate mineral which can be used include kaolinite, lizardite, halloysite and mixtures or combinations thereof, wherein kaolinite is sometimes preferred.

The particle size of the layer type 1:1 silicate mineral is not particularly limited, unless the resulting paste gets to inhomogeneous. The mean particle size is typically in a range between 0.01 and 100 μm or between 0.1 and 50 μm or between 1 and 25 μm.

The content of the layer type 1:1 silicate mineral in the composition is not particularly limited, unless the desired advantages cannot be obtained. If present, the layer type 1:1 silicate mineral is typically present in an amount from 3 wt. % to 55 wt. % or from 5 wt. % to 50 wt. % with respect to the whole composition.

Phyllosilicates from the layer type 2:1 silicate minerals which can be used include mica minerals, talc-pyrophyllite minerals, smectite minerals, vermiculite minerals, illites minerals.

Specific examples include talc, willemseite, pyrophyllite, stevensite, saponite (from the talc-pyrophyllite type group of minerals), stevensite, sponite, sauconite, hectorite, montmorillonite, beidellite, nontronite, volkonskite (from the smectite type group of minerals), phlogopite, biotite, lepidolite, muscovite, illite, glauconite, celadonite (from the mica type group of minerals).

The particle size of the layer type 2:1 silicate mineral is not particularly limited, unless the resulting composition gets too inhomogeneous. The mean particle size is typically between 0.01 and 100 μm or between 0.1 and 50 μm or between 1 and 25 μm.

The content of the layer type 2:1 silicate mineral in the composition is not particularly limited, unless the desired advantages cannot be obtained. If present, the layer type 2:1 silicate mineral is typically present in an amount from 3 wt. % to 55 wt. % or from 5 wt. % to 50 wt. % with respect to the whole composition

If a combination of layer type 1:1 silicate minerals and layer type 1:2 silicate minerals is used, the layer type 1:1 silicate mineral and the layer type 2:1 silicate mineral are typically present in the dental retraction composition in a certain weight ratio with respect to each other.

If a combination of phyllosilicate(s) is desired, the following mixtures can be used: layer type 1:1 silicates selected from kaolinite, lizardite, halloysite; layer type 1:2 silicates selected from mica minerals, talc-pyrophyllite minerals, smectite minerals, vermiculite minerals, illites minerals.

If present, the phyllosilicate(s) are typically present in the following amounts: Lower limit: at least 3 or at least 5 or at least 8 wt. %; Upper limit: utmost 60 or utmost 55 or utmost 50 wt. %; Range: from 3 to 60 or from 5 to 55 or from 8 to 50 wt. %; wt. % with respect to the dental retraction composition.

The dental composition comprises organic acid(s).

The organic acids are typically low-molecular organic acids and have a molecular weight in the range of 45 to 250 g/mol. The pKs-value of the organic acids is typically in the range of 2 to 5. The organic acids typically have 1 to 3 carboxylic acid moieties. The organic acids to be used are soluble in polyethylene glycol (Mw 400 g/mol), PEG 400. Soluble in polyethylene glycol means that at least 5 wt. % of the organic acids can be dissolved in PEG 400 at 23° C.

The solubility in polyethylene glycol is advantageous as it is an indicator for proving that the organic acid can easily be incorporated in the dental retraction composition, which is essentially free of water.

The pKs-value should not be too high to avoid damage to the sulcus and/or hard dental tissue of the patient to be treated.

Using organic acids with a rather low molecular weight was found be beneficial from a blood coagulation perspective.

The organic acids to be used should be non-toxic.

Suitable organic acids include tartaric acid, citric acid, gallic acid, pyruvic acid, succinic acid, glutaric acid, malic acid, acetic acid, formic acid and mixtures thereof.

The use of the following organic acids is sometime preferred: tartaric acid, citric acid and mixtures thereof.

The organic acid(s) is typically present in the following amounts: Lower limit: at least 1 or at least 5 or at least 8 wt. %; Upper limit: utmost 30 or utmost 25 or utmost 20 wt. %; Range: from 1 to 30 or from 5 to 25 or from 8 to 20 wt. %; wt. % with respect to the dental retraction composition.

The dental retraction composition comprises paste-forming component(s).

The nature and structure of the paste-forming component(s) is not particularly limited, either unless the desired result cannot be achieved.

Paste-forming component(s) include those, which are able to form a paste with the other components present.

The paste-forming component can typically be characterized by one or more of the following features: being non-aqueous; being hydrophilic; having a boiling point above 100° C.; typically having a molecular weight (Mw) in the range of 60 to 10,000 g/mol, or 200 to 10,000 g/mol.

A molecular weight as outlined above can be beneficial because the risk of an undesired evaporation of the paste forming liquid(s) from the dental retraction composition can be reduced.

The amount of paste-forming component in the composition is not particularly limited, unless the desired advantages cannot be obtained.

If the amount of paste-forming component in the composition is too low, the viscosity of the composition typically increases having the result that the extrusion force needed for dispensing the composition from a container might increase as well.

If the amount of paste-forming component in the composition is too high, the viscosity of the composition typically decreases having the result that the flow resistance might be insufficient and may hamper the application of the composition into the sulcus.

The paste-forming component(s) are typically present in the following amounts: Lower limit: at least 1 or at least 5 or at least 10 wt. %; Upper limit: utmost 60 or utmost 55 or utmost 50 wt. %; Range: from 1 to 60 or from 5 to 55 or from 10 to 50 wt. %; wt. % with respect to the dental retraction composition.

With increasing amount of paste-forming component(s), the viscosity of the dental retraction composition can be adjusted, in particular lowered. Good paste properties can be obtained, if the paste-forming component(s) are present in an amount of 10 to 60 wt. % or 10 to 50 wt. %.

Paste-forming components which can be used include components which do not comprise a radically-curable moiety, or components which comprise a radically-curable moiety and mixtures of both.

According to one embodiment, the dental retraction comprises paste-forming components not comprising a radically-curable moiety.

Examples of paste-forming components not comprising a radically-curable moiety include glycols (including ethylene glycol, propylene glycol, butylene glycol) and the respective alkyl ethers, block-co-polymers of ethylene glycol and propylene glycol (commercially available e.g., as Synperonic™ and Pluronic™, copolymers of ethylene glycol, propylene glycol and/or tetrahydrofuran, and alkoxylated glycerine or alkoxylated pentaerythritol or other multifunctional alcohols, glycerine and mixtures thereof.

In particular, the following paste-forming component were found to be useful: polyethylene glycol, polypropylene glycol and mixtures thereof.

If present, paste-forming component(s) not comprising a radically-curable moiety are typically present in the following amounts: Lower limit: at least 0.5 or at least 1 or at least 2 wt. %; Upper limit: utmost 60 or utmost 55 or utmost 50 wt. %; Range: 0.5 to 60 or 1 to 55 or 2 to 50 wt. %; wt. % with respect to the dental retraction composition.

According to one embodiment, the dental retraction composition comprises paste-forming components comprising a radically-curable moiety.

Paste-forming components comprising a radically-curable moiety can be characterized by the following features alone or in combination: comprising at least one (meth)acrylate moiety; comprising a (poly)oxy alkylene moiety; being hydrophilic.

Using components which are hydrophilic can be advantageous to facilitate an adequate moisture uptake of the composition during use.

Acrylate or methacrylate-functionalized monomers are suitable as basic components for free radical polymerization. Monomers can be mono-, di- or higher acrylate or methacrylate functionalized compounds and mixtures of monomers can be used.

Suitable examples of the paste-forming components comprising a radically-curable moiety include liquid (meth)acrylate functionalized homo- or copolymers of ethylene glycol, propylene glycol and/or THF and mixtures thereof.

Hydrophilic monomers which can also be used include acid functionalized monomers such as mono- or dimethacrylate-functionalized tartaric acid or citric acid. Such components may positively contribute to the haemostatic properties of the composition.

More specific examples can be characterized by the following general formula with n being in a range of 3 to 15.

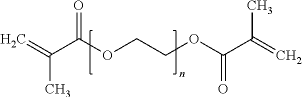

In particular, the following radically-curable components were found to be useful: polyethylene glycol dimethacrylate (Mₙ=330 g/mol), polyethylene glycol dimethacrylate (Mₙ=550 g/mol), polyethylene glycol dimethacrylate (Mₙ=750 g/mol), tetraethylene glycol dimethacrylate (Mₙ=302 g/mol), di-methacrylate functionalized copolymer of ethylene oxide and THF (Mₙ about 6,000 g/mol).

If present, the paste-forming components comprising a radically-curable moiety is present in the following amounts: Lower limit: at least 1 or at least 5 or at least 10 wt. %; Upper limit: utmost 50 or utmost 45 or utmost 40 wt. %; Range: from 1 to 50 or from 5 to 45 or from 10 to 40 wt. %; wt. % with respect to the total amount of the dental retraction composition.

The dental retraction composition may comprise an initiator or initiator system in addition.

An initiator or initiator system is typically present, if the dental retraction composition comprises paste-forming components with a radically-curable moiety.

Radically-curable components are typically cured by way of a free-radical polymerization, with the free radicals being provided via suitable initiator systems photochemically, and/or by redox reactions.

According to one embodiment, the dental retraction composition comprises a photo-initiator or photo-initiator system. The nature and structure of the photo-initiator system is not particularly limited unless the intended purpose is not negatively affected.

The photo-initiator is capable of generating free radicals for polymerization upon exposure to light. The photo-initiator(s) has typically a light absorption band in a wavelength range of 390 to 500 nm. Using a photo-initiator being soluble in the dental retraction composition described in the present text is preferred.

Photo-initiators which can be used typically contain a moiety selected form acyl phosphine oxids, benzoin ether, acetophenone, benzoyl oxime, phenylglyoxate, α-hydroxyketones or α-aminoketones.

A particularly suitable class of photo-initiators include the class of acylphosphine oxides, as described e.g. in U.S. Pat. No. 4,737,593 (Elrich et al.).

Such acylphosphine oxides can typically be characterized by the following formula

(R⁹)₂—P(═O)—C(═O)—R¹⁰

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S—, O—, or N-containing five- or six-membered heterocyclic group, or a —Z—C(═O)—P(═O)—(R⁹)₂ group, wherein Z represents a divalent hydrocarbyl group such as alkylene or phenylene having 2 to 6 carbon atoms.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms.

Suitable bisacylphosphine oxides can also be described by the following formula

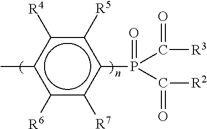

wherein n is 1 or 2, and R⁴, R⁵, R⁶ and R⁷ are H, C₁₋₄ alkyl, C₁₋₄ alkoxyl, F, Cl or Br; R² and R³, which are the same or different, stand for a cyclohexyl, cyclopentyl, phenyl, naphthyl, or biphenylyl radical, a cyclopentyl, cyclohexyl, phenyl, naphthyl, or biphenylyl radical substituted by F, Cl, Br, I, C₁₋₄ alkyl and/or C₁₋₄ alkoxyl, or an S or N-containing 5-membered or 6-membered heterocyclic ring; or R² and R³ are joined to form a ring containing from 4 to 10 carbon atoms and being optionally substituted by 1 to 6 C₁₋₄ alkyl radicals.

Examples include bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dimethylphenylphosphine oxide.

A preferred acylphosphine oxide is bis(2,4,6-trimethylbenzoyl)phenyl phosphine oxide (OMNIRAD™ 819, IGM Resin B. V., Waalwijk, Netherlands).

In another embodiment, it is preferred to use liquid blends of acylphosphine oxides with at least one other photo-initiator (available e.g. as OMNIRAD™ 1000, OMNIRAD™ 2022, OMNIRAD™ 2100 or OMNIRAD™ 4265, IGM Resin B. V., Waalwijk, Netherlands).

If present, the photo-initiator system is typically present in the following amounts: Lower amount: at least 0.01 or at least 0.05 or at least 0.1 wt. %; Upper amount: at most 8 or at most 5 or at most 4 wt. %; Range: 0.01 to 8 or 0.01 to 5 wt. % or 0.01 to 4 wt. %; wt. % with respect to the dental retraction composition.

If present, the photo-initiator is typically activated with light having a wavelength in the visible range (e.g. 430 to 480 nm). A light device which can be used is e.g. 3M's Elipar™ DeepCure-S LED Curing light (3M Oral Care).

The curing of the paste-forming components comprising radically-curable moieties can also be accomplished by using a redox-initiator system.

Using a redox-initiator system for curing the curable components alone or in combination with a photo-initiator system is sometimes preferred as the curing of the composition can also proceed in the dark, e.g. in those regions of the sulcus which are difficult to reach by light or if the dental retraction composition is not sufficiently transparent to light.

As the dental retraction composition comprises an organic acid and is thus acidic, the redox-initiator system should be able to function within an acidic environment.

In this respect redox-initiator systems comprising the following components were found to be useful: ascorbic acid component, organic peroxide, transition metal component, wherein the ascorbic acid component is stored separately from the transition metal component and the organic peroxide.

Alternatively, redox-initiator systems comprising the following components can also be used: salt of a barbituric acid or thiobarbituric acid component, peroxodisulfate or peroxodiphosphate component, sulfinic acid component, transition metal component(s), in particular copper components, wherein the salt of a barbituric acid or thiobarbituric acid component and the sulfinic acid component are stored separately from the other components of the redox-initiator system.

Ascorbic acid component which can be used include salts and esters of ascorbic acid, ethers, ketals, or acetals. Suitable salts include the alkali metal and earth alkali metal salts like Na, K, Ca and mixtures thereof. Esters of ascorbic acid include those which are formed by reacting one or more of the hydroxyl functions of ascorbic acid with a carboxylic acid, in particular the C₂ to C₃₀ carboxylic acid.

Suitable examples of C₂ to C₃₀ carboxylic acids include the fatty acids, like caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid.

Preferred are sometimes ascorbic components, which can be easily dissolved in or mixed with other components, which may be applied in combination with the ascorbic acid component, such as polymerizable components without acidic moieties. That is, using an ascorbic acid component having in addition a hydrophobic moiety can be preferred.

Suitable hydrophobic moieties include saturated and unsaturated aliphatic residues (e.g. C₂ to C₃₀ or C₁₂ to C₃₀). Those ascorbic acid derivatives may also function as surface-active substances (substances having a so-called “head/tail structure”). Particularly preferred are ascorbyl palmitate, ascorbyl stearate, mixtures and salts thereof.

The ascorbic acid component is typically applied together with one or more organic peroxides. Generally, all organic peroxide(s) can be used, if suitable to achieve the desired result.

In contrast to inorganic peroxides, organic peroxide(s) do not comprise metals or metal ions. Thus, organic peroxides typically only comprise C, O, H and optionally halogens (e.g. F, Cl, Br).

According to one embodiment, the organic peroxide is a di-peroxide, preferably a di-peroxide comprising the moiety R—O—O—R²—O—R³, with R¹ and R³ being independently selected from H, alkyl (e.g. C₁ to C₆), branched alkyl (e.g. C₁ to C₆), cycloalkyl (e.g. C₅ to C₁₀), alkylaryl (e.g. C₇ to C₁₂) or aryl (e.g. C₆ to C₁₀) and R² being selected from alkyl (e.g. (C₁ to C₆) or branched alkyl (e.g. C₁ to C₆). Examples of suitable organic di-peroxides include 2,2-Di-(tert.-butylperoxy)-butane and 2,5-Dimethyl-2,5-di-(tert-butylperoxy)-hexane and mixtures thereof.

According to another embodiment, the organic peroxide is a hydroperoxide, in particular a hydroperoxide comprising the structural moiety R—O—O—H with R being (e.g. C₁ to C₂₀) alkyl, (e.g. C₃ to C₂₀) branched alkyl, (e.g. C₆ to C₁₂) cycloalkyl, (e.g. C₇ to C₂₀) alkylaryl or (e.g. C₆ to C₁₂) aryl. Examples of suitable organic hydroperoxides include t-butyl hydroperoxide, t-amyl hydroperoxide, p-diisopropylbenzene hydroperoxide, cumene hydroperoxide, pinane hydroperoxide, p-methane hydroperoxide and 1,1,3,3-tetramethylbutyl hydroperoxide and mixtures thereof.

Suitable transition metal component(s) include organic and/or inorganic salt(s) selected from titanium, vanadium, chromium, manganese, iron, cobalt, nickel, copper and/or zinc, with copper and iron being sometimes preferred.

Useful salts include acetate(s), chloride(s), sulphate(s), benzoate(s), acetylacetonate(s), naphthenate(s), carboxylate(s), bis(1-phenylpentan-1,3-dione) complexes, salicylate(s), complexes with ethylenediaminetetraacetic acid of either of the transition metals and mixtures thereof.

If the transition metal component is a copper containing component, the copper containing component may have the general formula CuXₙ, wherein X represents an inorganic and/or organic ion, and n=1 or 2.

Typical examples of copper component(s) which can be used include salts and complexes of copper including copper acetate, copper chloride, copper benzoate, copper acetylacetonate, copper naphthenate, copper carboxylates, copper bis(1-phenylpentan-1,3-dione) complex (copper procetonate), copper salicylate, complexes of copper with thiourea, ethylenediaminetetraacetic acid and/or mixtures thereof. The copper compounds can be used in hydrated form or free of water. Especially preferred is copper acetate.

Examples of the salt of barbituric acid or thiobarbituric acid components include

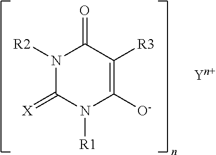

with R¹, R², and R³ being identical or different and being independently selected from hydrogen, alkyl, substituted alkyl, alkenyl, cycloalkyl, substituted cycloalkyl, arylalkyl, aryl or substituted aryl radical. R¹, R², and R³ may also contain a halogen radical such as chlorine or a hydroxyl, amino or nitro group.

If one of the radicals R¹ to R³ is an unsubstituted alkyl this radical can be straight-chain or branched and can contain, for example, from 1 to 18 carbon atoms, preferably from 1 to 10, and in particular from 1 to 6 carbon atoms. Examples of low-molecular alkyl radicals include methyl, ethyl, propyl, isopropyl, n-butyl, t-butyl, isobutyl, n-pentyl, and isoamyl.

If one of the radicals R¹ to R³ is a substituted alkyl radical then the alkyl moiety of this radical typically has the number of carbon atoms indicated above for unsubstituted alkyl. If one of the radicals R¹ to R³ is alkoxyalkyl or alkoxycarbonylalkyl then the alkoxy radical typically contains, for example, from 1 to 5 carbon atoms and is most typically methyl, ethyl, propyl, isopropyl, n-butyl, t-butyl, isobutyl, n-pentyl or isoamyl. If one of the radicals R¹ to R³ is alkenyl, it is typically a C₃ to C₅ alkenyl radical, especially allyl.

If one of the radicals R¹ to R³ is unsubstituted cycloalkyl, it is typically a C₄ to C₇ cycloalkyl radical, such as cyclopentyl or cyclohexyl. If one of the radicals R¹ to R³ is a substituted cycloalkyl then it is typically one of the above-indicated cycloalkyl radicals, with the substituent or substituents on the cycloalkyl radical possibly being, for example, C₁ to C₄ alkyl such as methyl, ethyl, propyl, n-butyl or isobutyl, fluoro, chloro, bromo, iodo or C₁ to C₄ alkoxy, especially methoxy. If one of the radicals R¹ to R³ is aryl or aralkyl, then it is typically a phenyl or naphthyl as aryl. Particularly suitable arylalkyl radicals are benzyl and phenylethyl.

R¹ to R³ may also be substituted aryl radicals if desired. In this case phenyl and naphthyl are preferred and as ring substituents C₁ to C₄ alkyl, especially methyl, halogen or C₁ to C₄ alkoxy, especially methoxy.

X is oxygen or sulfur.

Y may be an organic cation or an inorganic cation. The inorganic cation can be, but is not limited to, any metal M that is able to provide a cation M⁺, M²⁺, or M³⁺. Suitable inorganic cations include the cations of Li, Na, K, Mg, Ca, Sr, Ba, Al, Fe, Cu, Zn, or La.

The organic cation may also be a cation derived from ammonia or primary, secondary or tertiary amines. The substituents of primary, secondary or tertiary amines include alkyl, substituted alkyl, alkenyl, cycloalkyl, substituted cycloalkyl, arylalkyl, aryl or substituted aryl or mixtures thereof.

Specific examples of the barbituric acid components which can be used for producing the respective salt include barbituric acid, thiobarbituric acid, 1,3,5-trimethylbarbituric acid, 1-phenyl-5-benzylbarbituric acid, 1-benzyl-5-phenylbarbituric acid, 1,3-dimethylbarbituric acid, 1,3-dimethyl-5-phenylbarbituric acid, 1-cyclohexyl-5-ethylbarbituric acid, 5-laurylbarbituric acid, 5-butylbarbituric acid, 5-allylbarbituric acid, 5-phenylthiobarbituric acid, 1,3-dimethylthiobarbituric acid, trichlorobarbituric acid, 5-nitrobarbituric acid, 5-aminobarbituric acid, and 5-hydroxybarbituric acid.

Example of the peroxodisulfate components include ammonium, sodium, and potassium peroxodisulfate, wherein sodium peroxodisulfate is sometimes preferred.

Sulfinic acid components which can be used typically have the general formula R¹SOO—R², with R¹ being an alkyl, substituted alkyl, alkenyl, cycloalkyl, substituted cycloalkyl, arylalkyl, aryl or substituted aryl residual, and R²═H, metal, or an alkyl, substituted alkyl, alkenyl, cycloalkyl, substituted cycloalkyl, arylalkyl, aryl or substituted aryl moiety, wherein sodium benzol sulfinate and/or sodium toluene sulfinate is sometimes preferred.

If one of the radicals R¹ or R² is an unsubstituted alkyl then this radical can be straight-chain or branched and can contain, for example, from 1 to 18 carbon atoms, preferably from 1 to 10, and in particular from 1 to 6 carbon atoms. Examples of low-molecular alkyl radicals are methyl, ethyl, propyl, isopropyl, n-butyl, t-butyl, isobutyl, n-pentyl, and isoamyl.

If one of the radicals R¹ or R² is a substituted alkyl radical then the alkyl moiety of this radical typically has the number of carbon atoms indicated above for unsubstituted alkyl. If one of the radicals R¹ or R² is alkoxyalkyl or alkoxycarbonylalkyl then the alkoxy radical contains, for example, from 1 to 5 carbon atoms and is preferably methyl, ethyl, propyl, isopropyl, n-butyl, t-butyl, isobutyl, n-pentyl or isoamyl. If one of the radicals R¹ or R² is haloalkyl then the halo moiety is understood to be fluoro, chloro, bromo or iodo.

If one of the radicals R¹ or R² is alkenyl, then it is typically a C₃ to C₅ alkenyl radicals, especially allyl. If one of the radicals R¹ or R² is unsubstituted cycloalkyl, then it is typically C₄ to C₇ cycloalkyl radicals, such as cyclopentyl or cyclohexyl.

If one of the radicals R¹ or R² is substituted cycloalkyl, then it is typically one of the above-indicated cycloalkyl radicals, with the substituent or substituents on the cycloalkyl radical possibly being, for example, C₁ to C₄ alkyl such as methyl, ethyl, propyl, n-butyl or isobutyl, fluoro, chloro, bromo, iodo or C₁ to C₄ alkoxy, especially methoxy.

If one of the radicals R¹ or R² is aryl or aralkyl, then it is typically a phenyl or naphthyl as aryl. Preferred arylalkyl radicals include benzyl and phenylethyl.

R¹ or R² may also be substituted aryl radicals if desired. In this case phenyl and naphthyl are preferred and as ring substituents C₁ to C₄ alkyl, especially methyl, halogen or C₁ to C₄ alkoxy, especially methoxy.

Specific examples of the sulfinic acid component include benzene sulfinic acid, sodium benzene sulfinate, sodium benzene sulfinate dihydrate, sodium toluene sulfinate, formamidine sulfinic acid, sodium salt of hydroxymethane sulfinic acid, sodium salt of 2,5-dichlorobenzenesulfinic acid, 3-acetamido-4-methoxybenzenesulfinic acid. Particularly suitable compounds are sodium toluene sulfinate or sodium benzene sulfinate and their hydrates.

Suitable redox-initiator systems are also described in U.S. Pat. No. 6,953,535 B2 (Hecht et al.) or US 2017/0216152 A1 (Hecht et al.). The content of these references is herewith incorporated by reference.

As outlined above, if the dental retraction composition comprises a redox-initiator system, the oxidizing components and the reducing components are separated from each other during storage.

Additives, which can be present in the composition include colourant(s), pharmaceutical drug(s), rheological modifier(s), surfactant(s), flavouring agent(s), antioxidant(s) or stabilizer(s), anti-microbial agent(s), preserving agent(s), mixtures and combinations thereof.

There is no need for additives to be present, however, if one or more additives are present, they are typically present in an amount which supports the intended purpose.

According to one embodiment, the dental retraction composition has a colour which may allow an easy detection (especially in a patient's mouth compared to oral tissue and/or tooth substance) and control whether after the treatment all residues have been removed, in particular if the medical composition is used as dental retraction composition.

E.g., a blue, green or yellow colour may be suitable. However, in view of some new impression techniques like e.g. digital scanning, other colours might be preferred, in particular if the medical composition is used as dental retraction composition. Some techniques prefer colours that are less visible for the scanning instrument e.g. red or white. Colouring of the retraction device can be achieved by incorporating colorants or pigments (organic and inorganic) into the composition.

Examples of colourants which can be used include chinoline yellow dye (sicovit), chromophthalblue A3R, red iron oxide 3395, Bayferrox™ 920 Z Yellow, Neazopon™ Blue 807 (copper phthalocyanine-based dye), Helio™ Fast Yellow ER, Brilliant Blue FCF, Fast Green FCF and/or Orange Yellow S. Pigments or dyes which are stable under acidic conditions are preferred.

According to a further embodiment, pharmaceutical drugs can be added.

Pharmaceutical drugs might contribute or enhance a haemostatic effect, e.g. caused by the addition of an astringent. Pharmaceutical drugs which can be added include adrenaline, epinephrine, propylhexidrin, adrenochrom-monosemicarbazone, propylgallat, etamsylate, batroxobin, thrombin, fibrin, norepinephrine, noradrenalin, teryzolin, oxymetazolin and other beta-2 Sympathomimetika, According to a further embodiment, rheology modifiers can be added. Rheology modifiers might contribute to the viscosity and effect the rinsability. Rheology modifiers which can be added include silicone oil.

In another embodiment, the dental retraction composition may comprise one or more surfactants. Typical surfactants, which can be used, include anionic, cationic or non-ionic surfactants.

There is no need for a surfactant to be present at all. However, if a surfactant is present, it is typically present in an amount of up to 2 wt. % or up to 1 wt. % or up to 0.05 wt. %, with respect to the whole composition.

In another embodiment, the dental retraction composition may comprise a flavouring agent(s) to improve the taste and/or smell of the composition.

Typical flavouring agent(s), which can be used, include but are not limited to isoamylacetate (banana), benzaldehyde (bitter almond). Cinnamic aldehyde (Cinnamon), ethylpropionate (fruity), methyl anthranilate (Grape), mints (e.g. peppermints), limonene (e.g. Orange), allylhexanoate (pineapple), ethylmaltol (candy), ethylvanillin (Vanilla), methylsalicylate (Wintergreen).

There is no need for a flavouring agent to be present at all. However, if a flavouring agent is present, it is typically present in an amount of up to 3 wt. % or up to 0.1 wt. % of up to 0.01 wt. %, with respect to the whole composition.

The dental retraction composition may also comprise an antioxidant or stabilizer(s).

Stabilizer(s) which can be used often comprise a phenol moiety or phosphonic acid moieties.

Specific examples of stabilizer(s) which can be used include: p-methoxyphenol (MOP), hydroquinone monomethylether (MEHQ), 2,6-di-tert-butyl-4-methyl-phenol (BHT; Ionol), phenothiazine, 2,2,6,6-tetramethyl-piperidine-1-oxyl radical (TEMPO) Vitamin E; N,N′-di-2-butyl-1,4-phenylenediamine; N,N′-di-2-butyl-1,4-phenylenediamine; 2,6-di-tert-butyl-4-methylphenol; 2,4-dimethyl-6-tert-butylphenol; 2,4-dimethyl-6-tert-butylphenol and 2,6-di-tert-butyl-4-methylphenol; 2,6-di-tert-butylphenol; pentaerythritoltetrakis(3-(3,5-di-tert-butyl-4-hydroxyphenyl)propionate) (previously known as Irganox™ 1010); octyl-3,5-di-tert-butyl-4-hydroxy-hydrocinnamate; octadecyl 3-(3,5-di-tert-butyl-4-hydroxyphenyl)propionate; 1,3,5-trimethyl-2,4,6-tris(3,5-di-tert-butyl-4-hydroxybenzyl)benzene; 2,2′,4,4′-tetrakis-tert-butyl-3,3′-dihydroxybiphenyl; 4,4-Butylidenebis(6-tert-butyl-m-cresol); 4,4′-Isopropyliden-bis-(2-tert-butylphenol); 2,2′-methylenebis(6-nonyl-p-cresol); 1,3,5-tris(3,5-di-tert-butyl-4-hydroxybenzyl-)-1,3,5-triazine-2,4,6(1H,3H,5H)trione; pyrogallol; N-nitroso-N-phenylhydroxylamine; 3-propenylphenol, phenothiazine, N-phenyl-2-naphthylamine, phosphorous acid phenylphosphonic acid; vinylphosphonic acid or combinations or mixtures thereof.

If present, the additive(s) are typically present in the following amounts: Lower limit: at least 0.01 or at least 0.1 or at least 0.5 wt. %; Upper limit: utmost 20 or utmost 15 or utmost 10 wt. %; Range: 0.01 to 20 or 0.1 to 15 or 0.5 to 10 wt. %; wt. % with respect to the total amount of the dental retraction composition.

The dental retraction composition can also be characterized by its physical properties.

The dental retraction composition may be characterized by the following properties alone or in combination:
a) pH value: 2 to 5 if brought in contact with water,
b) water uptake: at least 150% with respect to the weight of the dental retraction composition,
c) gap resistance: at least 2.0 mm or at least 2.5 mm or at least 3.0 mm;
d) extrusion force: less than or equal to 150 N or below 140 N or below 130 N, e.g. if the dental retraction paste is dispensed from a container having a cannula with the dimension shown in FIG. 4 and using a piston as shown in FIGS. 5 and 6 of WO 2010/138433 A1 (3M);
e) rinsing time: less than or equal to 20 or less than or equal to 15 s;
f) flow resistance: 3 N or more,
g) depth of cure: at least 2 mm for dental retraction compositions comprising radically-curable components and an initiator system.

If desired, the respective features can be determined as described in the example section.

In certain embodiments, the combination of the following features is sometimes desirable: a) and b); b), c) and d); a), b) and e); a) and g); or a), b) and g).

The dental retraction composition can be produced by mixing the respective components.

If radically-curable components and a photo-initiator is present, the mixing should be conducted under save light conditions.

The dental retraction composition described in the present text may comprise, consist essentially of or consists of the following components:
guanidinyl-containing polymer in an amount of 1 to 60 wt. %,
carrageenan in an amount of 5 to 40 wt. %,
filler in an amount of 5 to 70 wt. %,
paste-forming component in an amount of 1 to 60 wt. %,
organic acid in an amount of 1 to 30 wt. %,
optionally radically curable components in an amount of 5 to 60 wt. %,
optionally a photo-initiator system in an amount of 0.1 to 8 wt. %,
additive(s) in an amount of 0 to 10 wt. %,
   wt. % with respect to the dental retraction composition.

The dental retraction composition may also comprise, consist essentially of or consists of the following components:
guanidinyl-containing polymer in an amount 5 to 40 wt. %,
carrageenan in an amount of 8 to 35 wt. %,
filler in an amount of 10 to 60 wt. %,
paste-forming component in an amount of 10 to 50 wt. %,
organic acid in an amount of 5 to 25 wt. %,
optionally radically curable components in an amount of 10 to 50 wt. %,
optionally a photo-initiator system in an amount of 0.1 to 8 wt. %,
additive(s) in an amount of 1 to 8 wt. %,
   wt. % with respect to the dental retraction composition.

The invention also relates to a delivery device comprising the dental retraction composition described in the present text.

During storage, the composition or paste described in the present text is typically packaged in a suitable packaging and delivery device.

The dental retraction composition is typically provided to the practitioner under hygienic conditions. One possibility to achieve this includes packing or storing the dental retraction composition in a sealed container such as a capsule, cartridge or foil bag under hygienic conditions.

A suitable container typically has a front end and a rear end, a piston movable in the container and a nozzle or cannula for delivering or dispensing the composition located in the container. The container has usually only one compartment or reservoir.

A suitable container may have a volume in the range of 0.1 to 1 ml. This is the volume typically needed for a single dental retraction procedure. Such a container is typically used only once (e.g. disposable packing).

If more than one retraction procedure should be done, a container may have a larger volume, e.g. in the range of 1 ml to 10 ml.

The dental retraction composition can be dispensed out of the container by moving the piston in the direction of the nozzle. The piston can be moved either manually or with the aid of an application device or applier designed to receive the container (e.g. an application device having the design of a caulk gun).

According to one embodiment, the dental retraction composition of the present text is stored in a one-compartment delivery device.

Examples of containers which can be used include compules, syringes and screw tubes. Containers of this kind are exemplified in more detail e.g. in U.S. Pat. No. 5,927,562 (Hammen et al), U.S. Pat. No. 5,893,714 (Arnold et al.) or U.S. Pat. No. 5,865,803 (Major).

It can be advantageous, if a container is used comprising a nozzle having a shape and size, which allows an easy and safe application of the dental retraction composition in the sulcus.

Useful containers typically have a hollow body (typically of cylindrical or conical shape) with a front end and a rear end in which the dental retraction composition is stored. The rear end is typically sealed with a piston, being movable in the hollow body. At the front end of the hollow body, there is typically a nozzle having a size and shape which enables the practitioner to dispense the medical composition into the sulcus of a patient. The smaller the diameter of the nozzle is, the easier the nozzle can be placed into the sulcus. However, a small diameter of the nozzle may result in an increase of the extrusion force needed to dispense the medical composition out of the device. Thus, not all cannula sizes and diameters are suitable. A device with a nozzle or cannula having an external diameter in the range from 0.6 mm to 1.3 mm and an internal diameter in the range from 0.2 mm to 0.9 mm has been found to be particular useful.

However, other shapes and diameters can be used as well, if the intended effect (i.e. widening of the sulcus) can be achieved.

It has been found that especially a container described in more detail in US 2011/151403 A (Pauser et al.) is useful for storing and dispensing the dental retraction composition described in the present text. If this particular combination is used, the dental retraction composition can be easily dispensed into the sulcus of a tooth and the desired retraction achieved.

The container which can advantageously be used for storing and dispensing the dental retraction composition comprises a cannula that has a free end which comprises an opening for dispensing the composition. Such a container facilitates the application of the dental retraction composition into the sulcus of a tooth in that it provides a mechanical means which allows an easy widening of the sulcus with the aid of the cannula. Once the sulcus has been widened, the dental retraction composition can easily be applied and due to its sufficient storage modulus may help stabilizing the widened sulcus.

In one embodiment the free end and the opening are shaped so that the opening can be positioned to the entry of the gingival sulcus, with an outer lateral surface of the free end touching the tooth and the gingiva. The free end is further preferably shaped so that the gingiva is laterally displaced, for example predominantly laterally displaced, from the tooth as the cannula is further moved with the opening toward the inside of the gingival sulcus. Thus, the cannula preferably allows for injecting the medical composition in a pre-opened gingival sulcus which may help to reliably fill the gingival sulcus with the medical composition.

In another embodiment the free end has an outer lateral surface which extends between a first outer diameter D1 and a second outer diameter D2. Preferably the first outer diameter D1 is located adjacent the front of the free end, or at the front most end. The second outer diameter D2 is preferably located at a distance L2 further to the rear from the first outer diameter D1. D2 is preferably greater than D1. This preferably enables the device to displace the gingiva laterally away from the tooth, and preferably thereby enables the device to widen the gingival sulcus as the free end is moved farther into the gingival sulcus. The term “diameter” may be generally interpreted as “cross-sectional dimension”, for cases in which a non-circular cross-section is provided.

The diameter D1 may be between 0.2 mm and 1 mm, in particular between 0.3 mm and 0.7 mm, or between 0.3 mm and 0.8 mm, in more particular D1 may be within a range of 0.4 mm to 0.6 mm. The diameter D1 is preferably about 0.4 mm. A relatively small dimension of the outer diameter D1 preferably allows, for example, the front of the free end to be inserted in the entry of the gingival sulcus relatively easily. Further such dimensions may help to reduce the risk of injuries of the gingival tissue during insertion of the front of the free end in the entry of the gingival sulcus, because it fits between the tooth and the gingiva rather than pressing on the gingiva itself.

The diameter D2 may be between 0.7 mm and 1.4 mm, in particular between 0.7 mm and 1.3 mm, in more particular the diameter D2 may be between 0.9 and 1.3 mm. Preferably the diameter D2 is about 1.1 mm. Such dimensions may for example provide the free end of the cannula with a sufficient stiffness, and on the other hand may still provide good interproximal access for the free end. Therefore, the device described in the present text may be suitable to inject a dental retraction composition in the gingival sulcus all around a tooth in a controlled manner, and not only at distal or lingual portions of the gingival sulcus. The length L2 of the free end may be between 0.3 mm and 2 mm, in particular between 0.3 mm and 1 mm, and preferably about 0.5 mm.

In another embodiment the first outer diameter D1 is located adjacent the opening. The first outer diameter D1 may also be formed by the opening. The opening may have a first inner diameter P1 which is between 0.2 mm and 1 mm, however the opening may further have a first inner diameter P1 which is between 0.3 mm and 0.7 mm. In particular P1 may be within a range of 0.4 mm to 0.6 mm, and preferably about 0.4 mm. P1 may be smaller than D1 but is preferably about equal to D1. In latter case P1 and D1 both refer to the diameter of the opening. In particular, the inner diameter P1 may provide for the flow rate of a high viscosity dental composition to be controlled relatively precisely as the composition is injected into the gingival sulcus.

In another embodiment the lateral outer surface of the free end tapers from the second outer diameter D2 toward the first outer diameter D1. Thus, the taper preferably tapers in a direction from D2 toward D1. Furthermore, the taper preferably tapers based on a curve having a relatively constant radius R. The Radius R may be greater than ½ of D2. For example, the shape of the free end may resemble a nose cone, a convex cone, or a radial cone. A curve resembling a radius greater than ½ of D2 may provide for a relatively low force required to insert the free end of the cannula in the entry of the gingival sulcus. Relative to a linear cone such convex or radial cone may further provide for a less blunt front-most end, which may reduce the risk of injuring the gingiva when inserted into the gingival sulcus.

The cannula of the container may have a length L1 between the first outer diameter D1 and a third outer diameter D3. The cannula may have a shaft portion extending between the second outer diameter D2 and the third outer diameter D3. The shaft portion and the free end may be located adjacent to each other, and together extend along the length L1. The third outer diameter D3 may be between 0.7 mm and 2 mm, in particular between 1.3 mm to 1.9 mm, and preferably about 1.7 mm. D3 is preferably greater than D2 but may also be about equal to D2. Thus, the shaft portion may be generally cylindrical or conical. Preferably the shaft portion smoothly transitions to the free end. The length L1 may be between 6 mm and 18 mm, in particular between 8 mm and 10 mm, and preferably about 9 mm. Such dimensions preferably allow the cannula to access areas that are accessible only through narrow spaces in a patient's mouth, for example a gingival sulcus between two teeth. This may also help in injecting a dental composition around substantially the entire perimeter of a tooth.

In one embodiment the cannula has a marking. The marking preferably is usable as reference with regard to a certain (for example a preferred) penetration depth of the cannula in the gingival sulcus. The marking may help a user to observe and/or to assess the depth to which the cannula is inserted in the gingival sulcus during a treatment of a patient. Therefore, a user may control the penetration depth of the cannula relatively precisely and thereby may achieve an effective gingival retraction. On the other hand this may help to avoid damage to the gingival tissue which may result from too deep penetration of the cannula in the gingival sulcus. The marking may be a notch, a rim, a step, or a (printed) line, for example. The marking may extend partly or entirely circumferentially around the cannula. The marking may further be formed by a transition between colors of outside surfaces of the cannula. For example, the front end of the cannula may have a certain first outside color, and an adjacent rear portion of the cannula may have a certain second outside color, wherein the first and second colors are different. The marking may also be formed by a transition between areas of different transparency or translucency. Preferably the marking is formed by a transition between surface structures of outside surfaces of the cannula. For example, the front end of the cannula may have a generally even or glossy outside surface, and an adjacent rear portion of the cannula may have a rougher or matt outside surface. The marking may also be a scale marking different penetration depths.

In one embodiment the container comprises a cartridge having a chamber for receiving and storing the dental retraction composition. The container is preferably adapted for comprising a piston or may comprise a piston. The container is preferably adapted for dispensing the dental retraction composition through the cannula. The cartridge may extend along a longitudinal axis, and the piston may be movable along the longitudinal axis for urging the dental retraction composition towards the cannula. The chamber may, for example open into a nozzle to which the cannula can be adapted. Alternatively, the chamber may open into the cannula. The cannula may be fixedly attached to the cartridge. For example, the cannula and the cartridge may be co-injection molded. In another embodiment the cannula and the cartridge are made from different plastic materials. For example, the cartridge may be made of a more rigid plastic material than the cannula. Therefore, the cartridge may provide sufficient stability for extruding the composition, and the cannula may be sufficiently soft to reduce the risk of injuries of the gingiva while in use.

In another embodiment the cannula may extend along a longitudinal axis which is inclined relative to the longitudinal axis of the cartridge by an angle of between 30 degrees and 60 degrees, preferably by about 45 degrees. The cannula may also extend along a curve, and a central axis through the opening of the cannula may be inclined relative to the longitudinal axis of the cartridge by an angle of between 30 degrees and 60 degrees, preferably by about 45 degrees.

In another embodiment the cannula comprises a passageway between the opening with the first inner diameter P1 and an inlet with a second inner diameter P2, wherein P2 is between 0.3 and 1.0 mm. P2 is preferably greater than or equal to P1. Thus, the passageway may taper towards the opening which may in dispensing certain dental compositions provide for a reduced extrusion force. Alternatively, the passageway may be generally cylindrical which may facilitate manufacturing.

Materials which can be used for producing the cannula include polyethylene, polypropylene, styrene-butadiene-styrene block copolymer, styrene-butadiene-methacrylate block copolymer, and thermoplastic polyurethane. Preferred plastic material for the container include polyamide, polyoxymethylene, polypropylene and polycarbonate.

According to one embodiment, the dental retraction composition described in the present text is stored in a two-compartments delivery device. Such a device is in particular useful for storing a dental retraction composition comprising curable components in combination with a redox-initiator system.

A suitable device for dispensing a dental material typically comprises a cartridge having two chambers for holding two components, and a mixer assembly, e.g. a mixer assembly comprising a housing that forms a mixing channel, a static mixing element being arranged within the mixing channel, wherein the mixer assembly further has an inlet end for receiving two components of a composition to be mixed and a dispensing end for dispensing the mixture of the components.

A suitable device for dispensing a dental composition may also comprise a container for containing the dental composition, the container having an outlet which is closed by a closure, a breakable seal connecting the closure and the container, a valve member, the device allowing for a relative movement between the valve member and the outlet between a storage position in which the outlet is closed and an operational position in which the outlet is open, wherein the valve member engages with the closure such that the relative movement between the valve member and the outlet also causes a relative movement between the closure and the outlet, whereby the seal is caused to break upon movement toward the operational position, and wherein the device is adapted to capture the closure in the device in the operational position.

Examples of suitable devices are described in US 2019/209268 A1 (Boehm), US 2019/298490 A1 (Boehm et al.) and U.S. Pat. No. 9,981,787 B2 (Boehm et al.).

The invention also relates to a kit of parts.

The kit of parts comprises the delivery device comprising the dental retraction composition described in the present text, an applier for the delivery device, optionally a dental impression material, optionally a retraction cap.

The dental impression material which can be used in combination with the dental retraction composition is not particularly limited in regard to their chemistry and nature. Polyether moieties or silicone moieties containing impression materials were found to be useful.

Examples of polyether moieties containing impression materials are given in U.S. Pat. No. 6,383,279, US 2002/0156149 and US 2005/02503871. Commercially available materials are sold e.g. under the brand Impregum™ (3M Oral Care).

Examples of silicone moieties containing impression materials are given in EP 1 893 163, US 2007/004858 and US 2006/293469. Commercially available materials are sold e.g. under the brand Imprint™ (3M Oral Care).

The kit may also comprise retraction caps. Retraction caps can be useful for keeping the dental retraction composition in place until an impression is taken or pushing the dental retraction composition into the sulcus. Retraction caps can be made of soft, tissue friendly material, e.g. cotton. However, other materials might be useful as well. If appropriate a temporary restoration can be used as retraction cap, too. Commercially available retraction caps are e.g. sold under the brand Comprecap™ (Coltene Whaledent).

In some cases compression caps or bridges, temporary crowns or bridges or even a first impression might be used as a kind of accessory during the retraction process. Typically, the dental retraction composition remains in the sulcus for a couple of minutes (e.g. 1 to 10 or 2 to 6 min to achieve effective mechanical retraction.

The kit may also comprise an applier or capsule dispenser. Those devices are commercially available e.g. from 3M Oral Care (cf. Product Catalogue 2007, page 29). Typical appliers have a gear ratio from about 3:1 to about 4:1. A further example of an applier, which can be used, is shown in U.S. Pat. No. 5,362,495 (Lesage), FIG. 3.

The dental retraction composition described in the present text is suitable for use in a method or retracting dental tissue of a tooth in the mouth of a patient.

As the dental retraction composition has astringent properties, besides its function as retraction material, the composition is also suitable for use in a method of coagulating blood in the sulcus of a tooth in the mouth of a patient.

More particularly, the dental retraction composition described in the present text is for use in a process of retracting dental tissue in combination with coagulating blood, wherein the process comprises the steps of
applying the dental retraction composition into the sulcus of a prepared tooth,
leaving the dental retraction composition in the sulcus for a time sufficient to absorb moisture and coagulating blood being present in the sulcus, e.g. for at least about 10 s or at least about 30 s or at least about 60 s,
optionally applying light to the dental retraction composition,
removing the dental retraction composition from the sulcus,
optionally making an impression of the hard dental tissue.

The removing of the dental retraction composition can be accomplished by different means.

If the retraction paste does not contain radically-curing components, the removing step is typically done by rising with water.

If the retraction paste contains radically-curing components and an initiator system, the paste can be cured by applying radiation or is self-curing. Once cured, these compositions can be removed ideally in one piece by using e.g. a tweezer or a probe.

Described is also a process of dispensing the dental retraction composition.

The process typically comprises the following steps:
providing a device or container containing the dental retraction composition as described in the present text,
placing the device or container in an applier or dispenser,
using the applier or dispenser for dispensing the dental retraction composition.

These steps can be repeated, if desired.

According to one embodiment, the dental retraction composition is inserted into the sulcus by the aid of the front end of the cannula of the container. This may facilitate the mechanically opening of the sulcus between soft and hard dental tissue.

A typical application procedure can be exemplified as follows: The dental retraction composition is dispensed by means of an applier out of a nozzle or cannula of a container into the sulcus of a prepared tooth structure of a mammal or human being. The dental retraction composition remains in the sulcus for an appropriate time period, which is typically determined by the practitioner.

After sufficient retraction, the dental retraction composition is removed from the sulcus using e.g. a dental water air syringe having sufficient pressure. Water-air beam devices are typically included in a dental chair.

The sulcus has been widened due to the application of the dental retraction composition compared to the sulcus before the application. After removal of the dental retraction composition, the shape of the prepared tooth including the preparation margin can be determined, either by an impression-taking process with a common impression material or by an intra-oral scan of the prepared region using e.g. an intra-oral scanner.

If desired the whole process and workability can also be demonstrated in vitro, e.g. using a Frasaco™ Standard Model AG3 (synthetic tissue surrounding an artificial tooth).

Further embodiments are described below:

Embodiment 1 relates to a dental retraction composition as described in the present text comprising or essentially consisting of or consisting of
filler(s) in an amount of 3 to 60 wt. %,
paste-forming component(s) not comprising a radically-curable moiety in an amount of 10 to 60 wt. %,
guanidinyl-containing polymer(s) in an amount of 1 to 60 wt. %;
carrageenan(s) in an amount of 1 to 40 wt. %,
organic acid in an amount of 1 to 40 wt. %,
additives in an amount of 0.1 to 10 wt. %,
wt. % with respect to the weight of the whole composition.

Embodiment 2 relates to a dental retraction composition as described in the present text comprising or essentially consisting of or consisting of
filler(s) in an amount of 3 to 60 wt. %,
paste-forming component(s) selected from glycol, glycerine, ethylene glycol, poly(ethylene glycol), propylene glycol, poly(propylene glycol), ethylene/propylene glycol co-polymers and mixtures thereof, and in an amount of 10 to 50 wt. %,
guanidinyl-containing polymer(s, the polymer being a polyethylene imine, and being present in an amount of 5 to 30 wt. %,
carrageenan(s) in an amount of 5 to 30 wt. %,
organic acid in an amount of 1 to 40 wt. %,
additives in an amount of 0.1 to 10 wt. %,
wt. % with respect to the weight of the whole composition.

Embodiment 3 relates to a dental retraction composition as described in the present text comprising or essentially consisting of or consisting of
phyllosilicate(s) selected from kaolinite, mica minerals and mixtures thereof and being present in an amount of 10 to 50 wt. %,
paste-forming component(s) selected from glycol, glycerine, ethylene glycol, poly(ethylene glycol), propylene glycol, poly(propylene glycol), and mixtures thereof, in an amount of 10 to 50 wt. %,
guanidinyl-containing polymer(s): the polymer being a polyethylene imine and being present in an amount of 5 to 30 wt. %,
carrageenan(s) being present in an amount of 5 to 30 wt. %,
organic acid in an amount of 1 to 40 wt. %,
additives in an amount of 0.1 to 10 wt. %,
wt. % with respect to the weight of the whole composition.

Embodiment 4 relates to a dental retraction composition as described in the present text comprising or essentially consisting of or consisting of
phyllosilicate(s) selected from kaolinite, mica minerals and mixtures thereof and being present in an amount of 10 to 50 wt. %,
paste-forming component(s) selected from glycol, glycerine, ethylene glycol, poly(ethylene glycol), propylene glycol, poly(propylene glycol), silicone oil and mixtures thereof, in an amount of 10 to 50 wt. %,
guanidinyl-containing polymer(s): the polymer being a polyethylene imine, and being present in an amount of 5 to 30 wt. %,
carrageenan(s) being present in an amount of 5 to 30 wt. %,
organic acid selected from tartaric acid, citric acid, gallic acid, pyruvic acid, succinic acid, glutaric acid, malic acid, acetic acid, formic acid and mixtures thereof, in an amount of 1 to 40 wt. %,
additives in an amount of 0.1 to 10 wt. %,
the dental retraction composition not comprising alone or in combination either of the following:
aluminium salt(s) such as aluminium chloride in an amount above 2 wt. %, water in an amount above 2 wt. %,
wt. % with respect to the weight of the whole composition.

Embodiment 5 relates to a dental retraction composition as described in the present text comprising or essentially consisting of or consisting of
filler(s) in an amount of 3 to 60 wt. %,
paste-forming component(s) comprising a radically-curable moiety in an amount of 10 to 60 wt. %,
guanidinyl-containing polymer(s) in an amount of 1 to 60 wt. %;
carrageenan(s) in an amount of 1 to 40 wt. %,
initiator for initiating a curing reaction of the component comprising a radically-curable moiety in an amount of 0.1 to 8 wt. %,
optionally organic acid in an amount of 1 to 40 wt. %,
additives in an amount of 0.1 to 10 wt. %,
wt. % with respect to the weight of the whole composition.

The following dental retraction compositions was found to be particularly useful:
A dental retraction composition comprising or essentially consisting of or consisting of guanidinyl-containing polymer selected from polymer(s) of the following formula

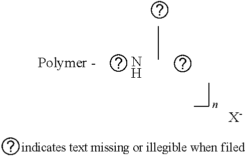

with X being selected from Cl⁻, Br⁻, I⁻, ½ SO₄²⁻, NO₃⁻, CH₃COO⁻, C₃H₇COO⁻, and n being a variable equal to at least 1 or in a range of 10 to 1,000, in an amount of 1 to 60 wt. %,
a carrageenan selected from iota, lambda carrageenan and mixtures in an amount of 5 to 40 wt. %,
filler selected from phyllosilicates and silica in an amount of 5 to 70 wt. %,
paste-forming component selected from either glycol, glycerine, ethylene glycol, poly(ethylene glycol), propylene glycol, poly(propylene glycol), copolymer(s) of ethylene glycol, propylene glycol and/or tetrahydrofuran and mixtures thereof, components comprising at least one (meth)acrylate moiety and a (poly)oxy alkylene moiety, and mixtures thereof, in an amount of 10 to 60 wt. %,
an organic acid selected from tartaric acid, citric acid, gallic acid, pyruvic acid, succinic acid, glutaric acid, malic acid, and mixtures thereof in an amount of 5 to 30 wt. %,
optionally an initiator system selected from a photo-initiator system, a redox-initiator system, and a combination of both in an amount of 0.1 to 8 wt. %,
additive(s) in an amount of 0 to 10 wt. %,
wt. % with respect to the weight of the whole composition.

The dental retraction composition described in the present text does typically not contain components producing a toxic, injurious, or immunological response in living tissue or components or additives which jeopardize the intended purpose to be achieved with the present invention.

Further, according to a specific embodiment, the dental retraction composition does typically not contain the following components alone or in combination: fibrillated fibres in an amount of more than 2 wt. %; starch or cellulose in an amount of more than 2 wt. %; water in an amount of more than 2 wt. %; aluminium chloride in an amount of more than 0.5 or 0.2 wt. %.

Fibrillated fibres are e.g. natural fibres based on cellulose or man-made fibres e.g. polyester, polyamide or fibres of glass.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate, but not limit, the scope of this invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Gap Resistance

The capability of a paste to open a sulcus and to keep a sulcus open can be determined by a device using a stamp which creates pressure created by a spring onto the curable paste in a small slit (residual gap device).

More precisely, the method can be described as follows: A mold having a rectangular shape with the dimensions: x (depth)=7.5 mm, y (width)=18.0 mm and z (height)=12.0 mm is provided.

The mold is formed by three immovable and one movable sidewall, all located on a plane surface. The movable sidewall is equipped with a spring having a defined spring pressure of 20N. The spring is compressed and fixed. The moveable sidewall is adjusted to a pre-defined depth 7.5 (cm).

2.0 g of the composition is mixed with 1.0 g either water or bovine blood on a mixing pad using a spatula. The mold is filled with the mixture 1 min after start of mix.

After a pre-defined time T1, the fixation of the spring is removed having the result that the spring exerts pressure on the curable composition through the movable sidewall. A portion of the curable composition is pressed out of the mold. The value x (cm) is decreasing.

After a pre-defined time T2, the value x (mm) is determined. The higher the value x at time T2 is, the higher the consistency of the composition is. For all results displayed below, T1=180 sec. from filling the gap with the paste and; T2=190 sec. from filling the gap with the paste

### Water Uptake

0.10 g of the paste is placed in 0.50 g water (app. 20° C.). The paste stays in contact with water for 2.0 min. Then the paste is removed and the weight of the swollen paste is measured on a scale. The water uptake is calculated as percentage increase of the final weight in relation to the original weight (0.10 g).

### Solubility in Polyethyleneglycol

The solubility of organic acids can be determined by dissolving 1 g of organic acid in 9 g of Polyethylene glycol (Mn=400 g/mol) for 30 min on a magnetic stirrer at 50° C. If the solution is clear and no residual solid components are visual, the organic acid is considered soluble in polyethylene glycol (Mn=400 g/mol). The organic acid has a solubility of at least 10%.

### Flow Resistance

The force needed to extrude a paste out of a defined mold is measured. The flow resistance is measured using a testing device Zwick Z020 machine (Zwick Roell Comp.). The testing device is equipped with a mold (diameter 8 mm, depth 5.6 mm) and a stamp (diameter 6 mm) to press the stamp against the paste inserted into the mold. The testing speed is set to 0.25 mm/s. The maximum force after 4 mm insertion is measured.

### Measurement of Extrusion Force

The extrusion force can be measured using as testing device a Zwick Z020 machine (Zwick Roell Comp.). The testing device is equipped with a holder for containers and a small stamp to press against the piston inserted in the container and sealing the reservoir. The dimensions of the stamp correspond to those used in commercially available single container dispensers (commercially available e.g. from 3M Oral Care.; order code 5706 SD). The feeding speed is set to 1.0 mm/s. The force is measured after the initial yield point was overcome (about 6-9 mm from starting point). The extrusion force was determined as an average value out of six individual measurements.

### Depth of Cure

If desired, the depth of cure can be determined in accordance with DIN EN ISO 6874:2015. The test is performed in a cylindrical metal form having a diameter of 4 mm and a length of 8 mm. Because of the elasticity of the resulting specimens instead of a measurement screw a ruler is used to measure the length of the specimen. The curing is done for 10 s using a 3M Elipar™ DeepCure L with a LED emitting at 430-480 nm and 1480 mW/cm². The measurement of the rinse time is also as described in C. Decoteau, M. Ogledzki, S. Soroushian, R. D. Perry, Rinse Time of Hemostatic Retraction Pastes, IADR 2011 #1025.

pH-Value

If desired, the pH value is determined by using a wet pH sensitive paper.

### Colour Change

2 g of the paste to be tested is mixed with 1 g bovine blood for 45 s on a mixing pad using a spatula. The colour of the mixture is inspected 1 min after start of mixing by an educated operator to judge on the change of colour compared to unmixed bovine blood.

### Materials

TABLE 1

| Component | Description |
|---|---|
| Guanidinylated polyethyleneimine | Guanidinyl-containing |
| (g-PEI), (preparation as described | polymer |
| in the experimental part of | |
| WO 2018/128704 A1) | |
| Iota-carrageenan | Carrageenan |
| Polyethylene glycol | Paste forming liquid 1 |
| (Mn = 400 g/mol) | (non-aqueous) |
| Mica; particle size: | Filler; layer type 1:2 |
| d70 = 1-3 μm | silicate mineral |
| Kaolin; particle size: | Filler, layer type 1:1 |
| d50 = about 14 μm | silicate mineral |
| Omnirad ™ 2022 (IGM Resins) | Photo-initiator |
| Di-methacrylate functionalized | Radically-curable |
| polyethylene oxide, Mn | component; Paste forming |
| about 750 g/mol) | liquid 2 (non-aqueous) |
| Tartaric acid, Gallic acid, | Organic acids |
| Citric Acid monohydrate | |

General Process for Producing the Dental Retraction Paste Paste forming liquid, guanidinylated polyethylene imine and organic acid were mixed. Then carrageenan was added and mixed. Filler was added and mixed. The initiator was added under save light conditions and mixed. All compositions were prepared in a vacuum speedmixer DAC-600.1 VAC at p<100 mbar.

### Compositions

The following compositions were prepared (Table 2):

TABLE 2

| | Ex. 1 | Ex. 2 | Ex. 3 | C.E. 1 | Ex. 4 |
|---|---|---|---|---|---|
| Tartaric Acid | 20 | | | | 17.5 |
| Gallic Acid | | 20 | | | |
| Citric Acid monohydrate | | | 20 | | |
| Polyethylene glycol | 28 | 28 | 28 | 38 | 5 |
| Poly(ethylene glycol) | | | | | 26 |
| dimethacrylate | | | | | |
| Guanylated Polyethyleneimine | 17 | 17 | 17 | 17 | 17 |
| Carrageenan | 17 | 17 | 17 | 17 | 17 |
| Filler | 18 | 18 | 18 | 28 | 17 |
| Photo-initiator | | | | | 0.5 |

The properties of the compositions were further evaluated (Table 3)

TABLE 3

| | Ex. 1 | Ex. 2 | Ex. 3 | C.E. 1 | Ex. 4 |
|---|---|---|---|---|---|
| Residual Gap with | 4.6 | 2.4 | 4.2 | 6.1 | 2.5 |
| 50 wt. % water [mm] | | | | | |
| Residual Gap with | 6.4 | 2.8 | 6.55 | 6.0 | 3.3 |
| 50 wt. % Blood [mm] | | | | | |
| Difference in Residual | 1.8 | 0.4 | 2.35 | −0.1 | 0.8 |
| Gap: water vs. blood | | | | | |
| Flow resistance [N] | 5.0 | 12.0 | 6.0 | 8.6 | 27.8 |
| Depth of cure [mm] | n.a. | n.a. | n.a. | n.a. | 3.0 |
| Color change with | Yes; | Yes; | Yes; | No; | Yes; |
| bovine blood | dark | brown | dark | slightly | brown |
| | brown | | brown | darker red | |

Pastes containing organic acids and about 50% bovine blood showed blood coagulation and an increase in Residual Gap Values compared to pastes containing 50% water. Further a rapid colour change after mixture with bovine blood was observed. Comparative Examples (C.E.) containing no organic acid showed no difference in Residual Gap Values between water and blood and no rapid colour change.

1. A dental retraction composition comprising
guanidinyl-containing polymer,
carrageenan,
filler,
paste-forming component,
organic acid having a molecular weight of 45 to 250 g/mol, a pks value of 2 to 5, 1 to 3 carboxylic acid moieties, and being soluble in polyethylene glycol with a molecular weight of 400 g/mol,
the dental retraction composition not containing water in an amount of more than 5 wt. %, and not containing aluminium chloride in an amount of more than 0.5 wt. %,
wt. % with respect to the weight of the dental retraction composition.

2. The dental retraction composition according to claim 1, the guanidinyl-containing polymer being characterized by the following formula: 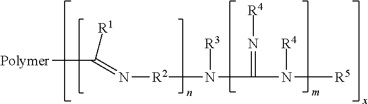
wherein R¹ is hydrogen, C₁-C₁₂ (hetero)alkyl, a C₅-C₁₂ (hetero)aryl, or a residue of the polymer chain; R² is a covalent bond, a C₂ to C₁₂ (hetero)alkylene, or a C₅-C₁₂ (hetero)arylene; R³ is hydrogen, C₁-C₁₂ (hetero)alkyl, C₅-C₁₂ (hetero)aryl, or a residue of the polymer chain when n is 0; each R⁴ is independently hydrogen, C₁-C₁₂ (hetero)alkyl, C₅-C₁₂ (hetero)aryl; R⁵ is hydrogen, C₁-C₁₂ (hetero)alkyl, or C₅-C₁₂ (hetero)aryl, or —N(R⁴) 2; n is 0 or 1; m is 1 or 2; and x is an integer equal to at least 1.

3. The dental retraction composition according to claim 1, the guanidinyl-containing polymer being selected from
polymer(s) having pendent or catenary guanidinyl groups of the following formula: 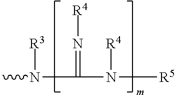
wherein, m is equal to 1 or 2 and the groups R³, R⁴, and R⁵ are the same as defined above or
polymer(s) of the following formula 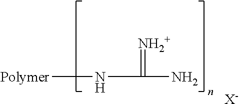
with X⁻ being selected from Cl⁻, Br⁻, I⁻, ½SO₄²⁻, NO₃⁻, CH₃COO⁻, C₃H₇COO⁻, and n being a variable equal to at least 1 or in a range of 10 to 1,000.

4. The dental retraction composition according to claim 1, the carragenan being selected from iota, lambda carrageenan and mixtures thereof.

5. The dental retraction composition according to claim 1, the organic acid being selected from tartaric acid, citric acid, gallic acid, pyruvic acid, succinic acid, glutaric acid, malic acid, acetic acid, formic acid and mixtures thereof.

6. The dental retraction composition according to claim 1, the paste-forming component being characterized by the following properties: being non-aqueous, being hydrophilic, and having a boiling point above 100° C.

7. The dental retraction composition according to claim 1, wherein the paste-forming component does not comprise a radically-curable moiety and is preferably selected from glycol, glycerine, ethylene glycol, poly(ethylene glycol), propylene glycol, poly(propylene glycol), butylene glycol, copolymer(s) of ethylene glycol, propylene glycol and/or tetrahydrofuran, alkoxylated glycerine or alkoxylated pentaerythritol and mixtures thereof.

8. The dental retraction composition according to claim 1, wherein the paste-forming component comprises at least one radically-curable moiety, and wherein the dental retraction composition optionally comprises in addition an initiator system, preferably a photo-initiator system.

9. The dental retraction composition according to claim 1, wherein the paste-forming component comprises a radically-curable moiety and is preferably characterized by the following features: comprising at least one (meth)acrylate moiety and comprising a (poly)oxy alkylene moiety.

10. The dental retraction composition according to claim 1, the paste-forming component being selected from 2-hydroxylethylmethacrylate, (meth)acrylate functionalized homo- or copolymers of ethylene glycol, propylene glycol and/or THF, mono- or di(meth)acrylate-functionalized tartaric acid or citric acid and mixtures thereof.

11. The dental retraction composition according to claim 1 comprising
guanidinyl-containing polymer in an amount of 1 to 60 wt. %,
carrageenan in an amount of 5 to 40 wt. %,
filler in an amount of 5 to 70 wt. %,
paste-forming component in an amount of 1 to 60 wt. %,
organic acid in an amount of 1 to 30 wt. %,
optionally an initiator system in an amount of 0.1 to 8 wt. %,
additive(s) in an amount of 0 to 10 wt. %,
wt. % with respect to the dental retraction composition.

12. The dental retraction composition according to claim 1 comprising
guanidinyl-containing polymer selected from polymer(s) of the following formula 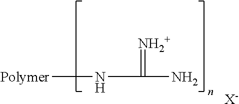
with X⁻ being selected from Cl⁻, Br⁻, I⁻, ½SO₄²⁻, NO₃⁻, CH₃COO⁻, C₃H₇COO⁻, and n being a variable equal to at least 1 or in a range of 10 to 1,000,
a carrageenan selected from iota, lambda carrageenan and mixtures in an amount of 5 to 40 wt. %,
filler selected from phyllosilicates in an amount of 5 to 70 wt. %, paste-forming component selected from
either glycol, glycerine, ethylene glycol, poly(ethylene glycol), propylene glycol, poly(propylene glycol), copolymer(s) of ethylene glycol, propylene glycol and/or tetrahydrofuran and mixtures thereof,
components comprising at least one (meth)acrylate moiety and a (poly)oxy alkylene moiety,
and mixtures thereof,
in an amount of 10 to 60 wt. %,
an organic acid selected from tartaric acid, citric acid, gallic acid, pyruvic acid, succinic acid, glutaric acid, malic acid, and mixtures thereof in an amount of 5 to 30 wt. %,
optionally an initiator system selected from a photo-initiator system, a redox-initiator system, and a combination of both in an amount of 0.1 to 8 wt. %,
additive(s) in an amount of 0 to 10 wt. %,
wt. % with respect to the weight of the whole composition.

13. The dental retraction composition according to claim 1 for use in a process of retracting dental tissue in combination with coagulating blood, the process comprising the steps of
applying the dental retraction composition into the sulcus of a prepared tooth,
leaving the dental retraction composition in the sulcus for a time sufficient to absorb moisture and to coagulate blood being present in the sulcus,
optionally applying light to the dental retraction composition,
removing the dental retraction composition from the sulcus.

14. (canceled)

15. A kit of parts comprising the dental retraction composition of claim 1, a delivery device a capsule, a compule, a syringe, a catride, or a combination thereof, an applier for the delivery device,
optionally a dental impression material, optionally a retraction cap.
